(19) United States
(12) Patent Application Publication
(10) Pub. No.: US 2022/0356147 A1
(43) Pub. Date: Nov. 10, 2022

(54) COMPOUNDS AND METHODS FOR POTENTIATING COLISTIN ACTIVITY

(71) Applicant: University of Notre Dame du Lac, South Bend, IN (US)

(72) Inventors: Christian Melander, South Bend, IN (US); Alexander WEIG, South Bend, IN (US); Akash Kumar BASAK, South Bend, IN (US)

(73) Assignee: University of Notre Dame du Lac, South Bend, IN (US)

(21) Appl. No.: 17/771,896

(22) PCT Filed: Oct. 8, 2020
(86) PCT No.: PCT/US2020/054733

§ 371 Date: Apr. 26, 2022

Related U.S. Application Data
(60) Provisional application No. 62/929,723, filed on Nov. 1, 2019.

(51) Int. Cl. C07C 235/64; A61P 31/04

(52) U.S. Cl. CPC C07C 235/64; A61P 31/04

(57) ABSTRACT

Infections caused by multidrug-resistant (MDR) bacteria, particularly Gram-negative bacteria, are an escalating global t health threat. Often clinicians are forced to administer the last resort antibiotic colistin, however colistin resistance is becoming increasingly prevalent, giving rise to the potential for a situation in which there are no treatment options for MDR Gram-negative infections. The development of adjuvants that circumvent bacterial resistance mechanisms is a promising orthogonal approach to the development of new antibiotics. We recently disclosed that the known IKK-13 inhibitor IMD-0354 potently suppresses colistin resistance in several Gram-negative strains. In this disclosure, we explore the structure activity relationship (SAR) between the IMD-0354 scaffold and colistin resistance suppression, and identify several compounds with more potent activity than the parent against highly colistin resistant strains of Acinetobacter baumannii and Klebsiella pneumoniae.

### RELATED APPLICATIONS

This application claims priority under 35 U.S.C. §119(e) to U.S. Provisional Patent Application No. 62/929,723, filed Nov. 1, 2019, which is incorporated herein by reference.

### GOVERNMENT SUPPORT

This invention was made with government support under Grant No. AI136904 awarded by the National Institutes of Health. The government has certain rights in the invention.

### BACKGROUND OF THE INVENTION

The threat to modern medicine caused by rising antibiotic resistance cannot be understated. Of all bacteria, the ESKAPE pathogens (Enterococcus faecium, Staphylococcus aureus, Kleb-siella pneumoniae, Acinetobacter baumannii, Pseudomonas aeru-ginosa, and Enterobacter species) are considered to pose the highest threats to human health. Despite escalating incidence of infections that stem from multidrug-resistant (MDR) strains of these pathogens, the antibiotic discovery pipeline to counter these threats still remains significantly underpopulated. In the case of the Gram-positive members of this group (E. faecium and S. aureus), limited success has been achieved with the introduction of linezolid and daptomycin over the last 20 years. For Gram-negative bacteria, however, the last class of novel antibiotics to be introduced into the clinic was the fluoroquinolone class in the 1960s.

As a result of this current antibiotic landscape, clinicians have become more reliant upon polymyxin antibiotics for the treatment of MDR Gram-negative bacterial infections.

Polymyxins are cationic cyclic peptides whose bactericidal activity is driven by physical disruption of the Gram-negative bacterial membrane. As an antibiotic for human use, colistin was introduced into the clinic in 1955 but rapidly fell out of favor due to nephrotoxicity, and was replaced by antibiotics with better safety profiles. However, with limited therapeutic options currently available, coupled with a historically low incidence of resistance due to limited use, colistin is now typically viewed as the antibiotic of last resort for the treatment of MDR Gram-negative bacterial infections.

As colistin treatment has become more common, there has been a corresponding upsurge in colistin resistance observed in the clinic over the past decade. Clinical resistance to colistin, and other polymyxins, is driven by cationic modification of the lipid A component of lipopolysaccharide (LPS) that decorates the outer membrane of Gram-negative bacteria. In A. baumannii, lipid A is typically modified with phosphoethanol-amine, while in K. pneumoniae it is typically modified with aminoarabinose. In either case, modification of lipid A serves to decrease the overall net negative charge of LPS, which, in turn, impacts the affinity of colistin for the outer membrane. Historically, colistin resistance has typically been mediated by chromosomal mutations in genes encoding two-component systems (such as PmrAB in A. baumannii) that, combined with colistin's limited use, meant dissemination of colistin-resistant isolates was slow. The situation changed dramatically with the emergence of the plasmid-borne colistin resistance genes mcr-1-9 that have raised the possibility of rapid dissemination of colistin resistance into the general pathogen pool via horizontal gene transfer, which would ultimately deprive clinicians of this last resort antibiotic.

With the rising incidence of MDR bacterial infections coupled with a depleted antibiotic pipeline, research into alternatives to traditional antibiotics is warranted. Accordingly, there is a need for compounds that circumvent bacterial resistance mechanisms against existing and new antibiotics.

### SUMMARY

The exploration into the structure—activity relationship (SAR) of the IMD-0354 scaffold and the identification of 35 compounds with equivalent or superior activity to IMD-0354 against both the representative strains used in the screen are described herein.

Accordingly, disclosure provides a compound of Formula I:

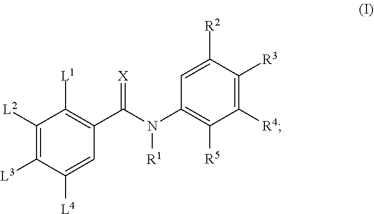

wherein

L¹ is OH, H, SH, NH₂, or —O(C═O)(C₁-C₈)alkyl wherein the moiety (C₁-C₈)alkyl is interrupted optionally with one or more heteroatoms;

L² is H, OH, or halo;

L³ is H, OH, or CF₃;

L⁴ is halo, H, or OH;

R¹ is H or —(C₁-C₆)alkyl;

R² is H, halo, CF₃, or —(C₁-C₆)alkyl;

R³ is CF₃, H, or halo;

R⁴ is halo, H, CF₃, NH₂, NO₂, or —(C₁-C₆)alkyl;

R⁵ is H, halo, or CF₃; and

X is O or S;
wherein at least one of L¹, L², L³ and L⁴ is OH or —O(C═O)(C₁-C₈)alkyl; and
wherein at least one of R², R³, R⁴ and R⁵ is not H.

This disclosure also provides a composition comprising the above and a pharmaceutically acceptable buffer, carrier, diluent, or excipient.

Additionally, this disclosure provides a method for treating a bacterial infection in a subject in need thereof comprising administering to the subject, concurrently or sequentially, a therapeutically effective dose of an adjuvant and a therapeutically effective dose of Colistin, wherein the adjuvant is the compound or composition disclosed above, and the bacterial infection is thereby treated.

The invention provides novel compounds of Formulas I, Ia, Ib, IIa, IIb, IIIA, Mb, IV, Va, Vb, VIa and VIb, intermediates for the synthesis of compounds of said Formulas, as well as methods of preparing compounds of said Formulas. The invention also provides compounds of said Formulas that are useful as intermediates for the synthesis of other useful compounds. The invention provides for the use of compounds of said Formulas for the manufacture of medicaments useful for the treatment of bacterial infections in a mammal, such as a human.

The invention provides for the use of the compositions described herein for use in medical therapy. The medical therapy can be treating bacterial infections. The invention also provides for the use of a composition as described herein for the manufacture of a medicament to treat a disease in a mammal, for example, a multi-drug resistant (MDR) Gram-negative bacterial infection in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Time-kill curves for compound 22 and colistin in AB 4106. Blue: Untreated bacteria. Red: 5 μM 22. Orange 5 μM 22+0.5 μg/mL colistin. Green: 5 μM 22+4 μg/mL colistin. Purple: 5 μM 22+32 μg/mL colistin. Data shows SD from at least four replicates.

FIG. 2. Time-kill curves for compound 22 and colistin in KP B9. Blue: Untreated bacteria. Red: 5 μM 22. Orange 5 μM 22+0.5 μg/mL colistin. Green: 5 μM 22+4 μg/mL colistin. Purple: 5 μM 22+32 μg/mL colistin. Data shows SD from at least four replicates.

FIG. 3. Dose response curves for cytotoxicity assay for compound 21.

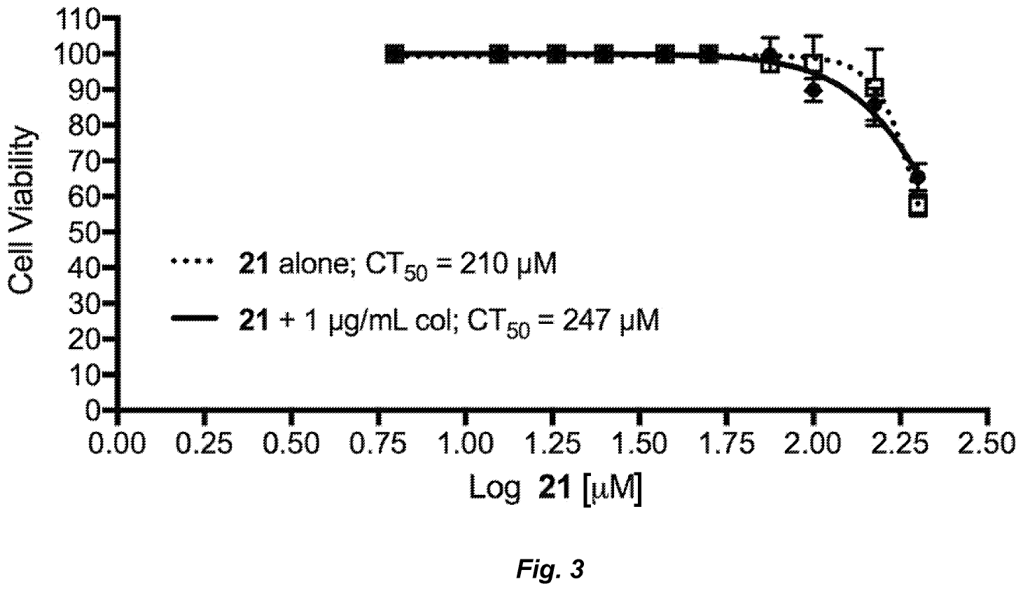

FIG. 4. Dose response curves for cytotoxicity assay for compound 25.

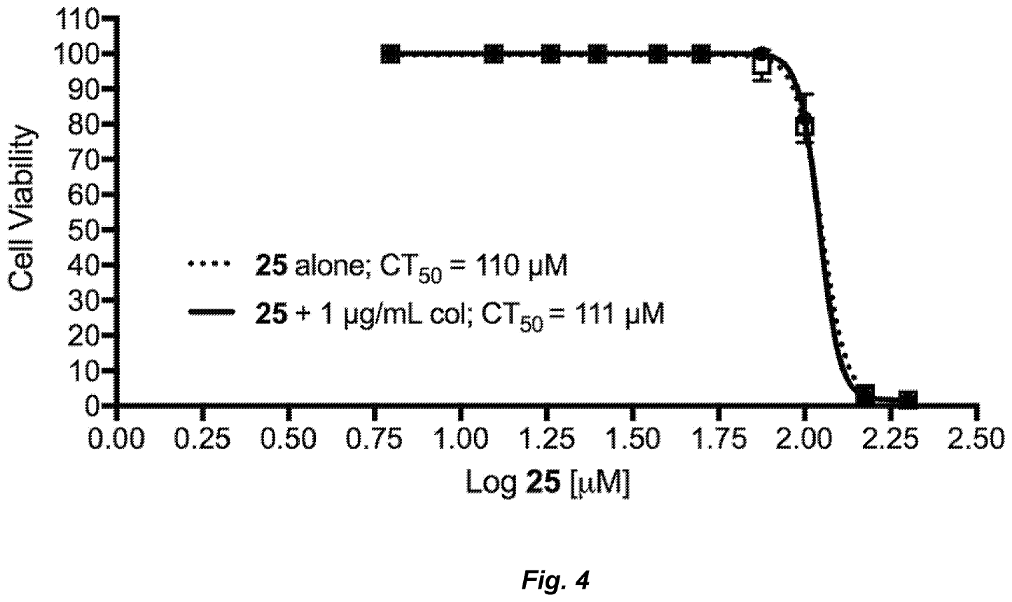

### DETAILED DESCRIPTION

Herein is disclosed the development of small molecule antibiotic adjuvants. Such molecules typically possess little stand-alone microbicidal activity, but instead target antibiotic resistance mechanisms. When paired with the appropriate antibiotic, these antibiotic/adjuvant combinations provide a powerful treatment for MDR bacteria. In the context of colistin resistance, we have identified a number of adjuvants capable of reversing colistin resistance in bacterial strains that contain chromosomally-encoded resistance mechanisms as well as those that harbor the plasmid-borne mcr-1 gene (Compounds A-E, Scheme 1). We showed that the non-toxic salicylamide IMD-0354 E, a well-known inhibitor of IKK-β, reversed lipid A modification and potently suppressed colistin resistance in A. baumannii, K. pneumoniae, and Escherichia coli, reducing the minimum inhibitory concentration (MIC) of colistin upwards of 4096-fold at 5 μM (1.9 μg/mL). Given this potency, we became interested in delineating the structural parameters of this scaffold that drive colistin potentiation.

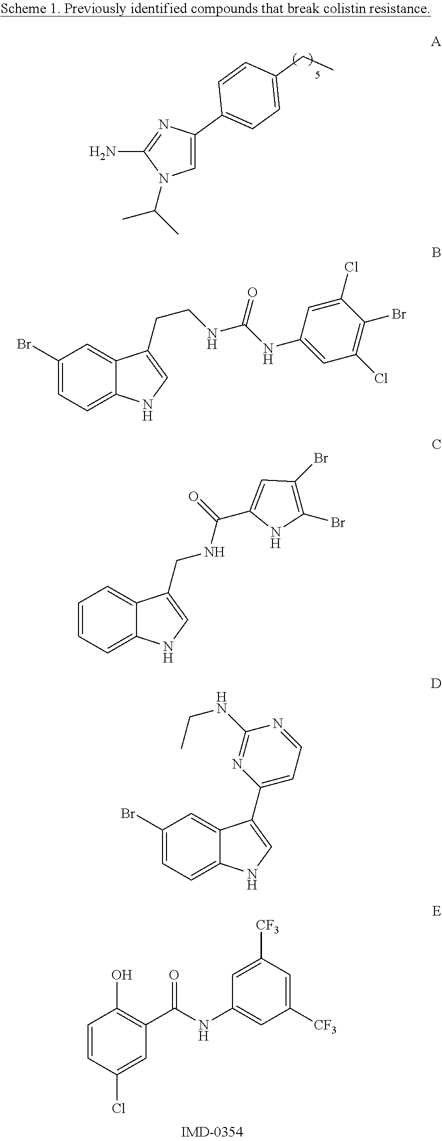

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “numberl” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated.

As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art.

However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-l-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)2, CN, CF3, OCF3, R′, O, S, C(O), S(O), methylenedioxy, ethylenedioxy, N(R′)2, SW, SOR′, SO₂R′, SO₂N(R′)2, SO₃R′, C(O)R′, C(O)C(O)R′, C(O)CH₂C(O)R′, C(S)R′, C(O)OR′, OC(O)R′, C(O)N(R′)2, OC(O)N(R′)2, C(S)N(R′)2, (CH₂)0-2NHC(O)R′, N(R′)N(R′)C(O)R′, N(R′)N(R′)C(O)OR′, N(R′)N(R′)CON(R′)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

The term “IC₅₀” is generally defined as the concentration required to kill 50% of the cells in 24 hours.

The term “adjuvant”, as used herein, refers to a pharmacological agent such as a small molecule compound that restores the activity of antibacterial agent that has been rendered less active or inactive against a bacterial infection in a subject due to drug-resistance.

### Embodiments of the Invention

This disclosure provide a compound of Formula I:

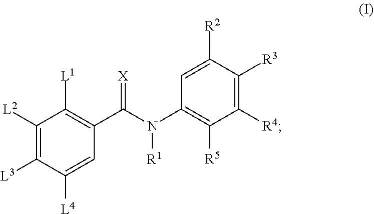

wherein

L¹ is OH, H, SH, NH₂, or —O(C═O)(C₁-C₈)alkyl wherein the moiety (C₁-C₈)alkyl is interrupted optionally with one or more heteroatoms;

L² is H, OH, or halo;

L³ is H, OH, or CF₃;

L⁴ is halo, H, or OH;

R¹ is H or —(C₁-C₆)alkyl;

R² is H, halo, CF₃, or —(C₁-C₆)alkyl;

R³ is CF₃, H, or halo;

R⁴ is halo, H, CF₃, NH₂, NO₂, or —(C₁-C₆)alkyl;

R⁵ is H, halo, or CF₃; and

X is O or S;

wherein at least one of L¹, L², L³ and L⁴ is OH or —O(C═O)(C₁-C₈)alkyl; and wherein at least one of R², R³, R⁴ and R⁵ is not H.

In some embodiments, the invention provides compounds, compounds of compositions and methods where a compound, compound of the composition or method is not one or more compounds disclosed in herein.

In various embodiments, R¹ is H and X is O. In other embodiments, the compound of

Formula I is represented by Formula Ia or Ib:

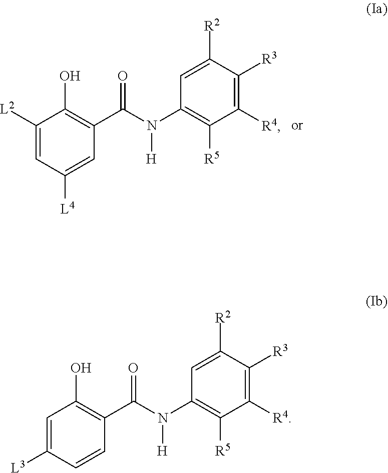

In other embodiments, the compound of Formula I is represented by Formula IIa or IIb:

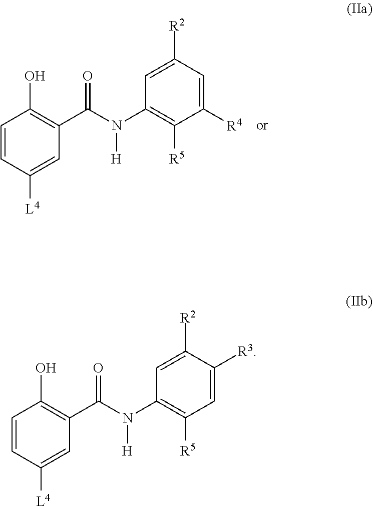

In other embodiments, the compound of Formula I is represented by Formula IIIa or IIIb:

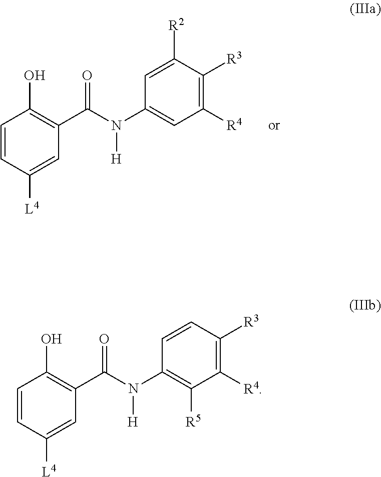

In other embodiments, the compound of Formula I is represented by Formula IV:

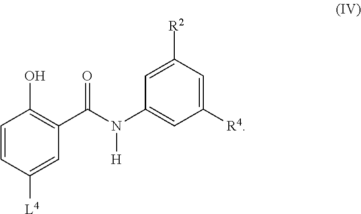

In other embodiments, the compound of Formula I is represented by Formula Va or Vb:

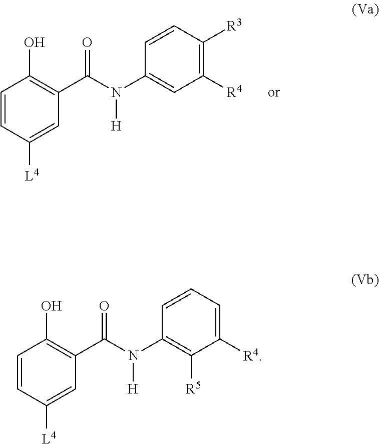

In other embodiments, the compound of Formula I is represented by Formula VIa or VIb:

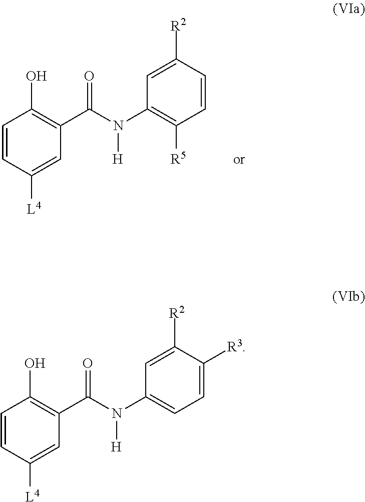

In various embodiments, at least one other of L¹, L², L³ and L⁴ is not H. In various embodiments, L⁴ is halo. In various embodiments, at least two of R², R³, R⁴ and R⁵ are not H. In various embodiments, at least two of R², R³, R⁴ and R⁵ comprises a halo substituent. In various embodiments, at least two of R², R³, R⁴ and R⁵ are halo. In various embodiments, at least two of R², R³, R⁴ and R⁵ are fluoro. In various embodiments, at least two of R², R³, R⁴ and R⁵ are chloro. In various embodiments, at least two of R², R³, R⁴ and R⁵ are bromo. In various embodiments, the compound comprises at least one iodo substituent. In various embodiments, at least two of R², R³, R⁴ and R⁵ are CF_{3.} In various embodiments, at least one of R², R³, R⁴ and R⁵ is halo and the other is CF_{3.}

In additional embodiments, the compound is:

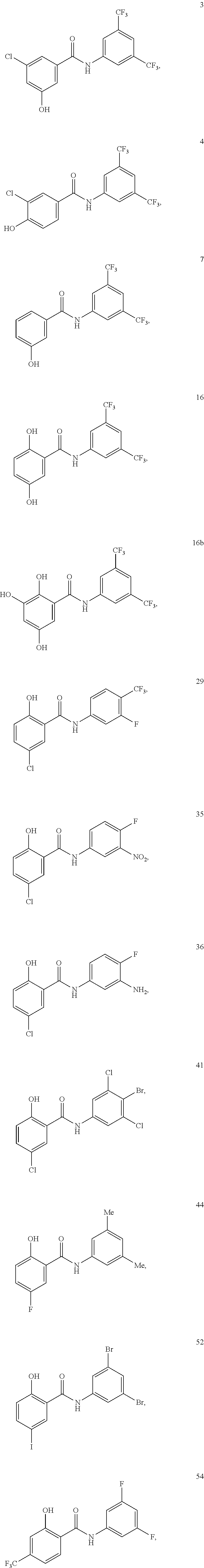

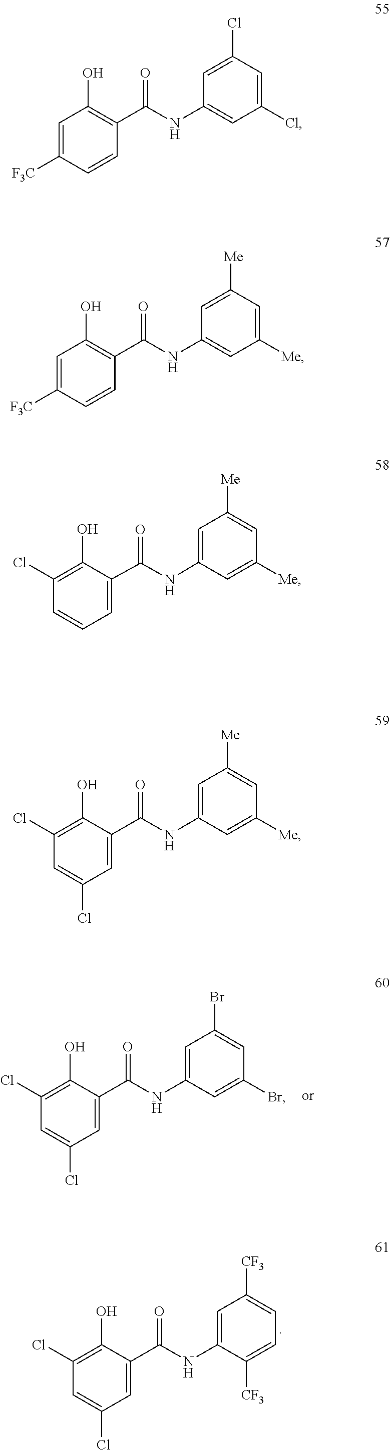

In other additional embodiments, the compound is:

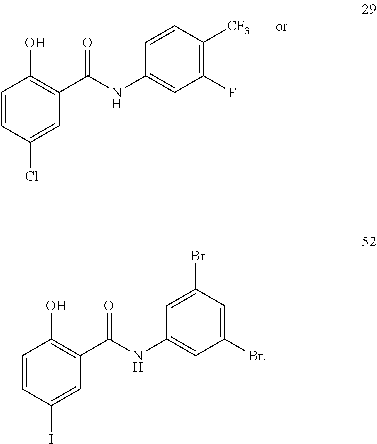 [SMILES: O=C(Nc1ccc(C(F)(F)F)c(F)c1)c1cc(Cl)ccc1O ; O=C(Nc1cc(Br)cc(Br)c1)c1cc(I)ccc1O]

This disclosure also provides a (pharmaceutical) composition comprising any one or more of the compounds above and a pharmaceutically acceptable buffer, carrier, diluent, or excipient.

Additionally, this disclosure provides a method for treating a bacterial infection in a subject in need thereof comprising administering to the subject, concurrently or sequentially, a therapeutically effective dose of an adjuvant and a therapeutically effective dose of an antibiotic or antibacterial agent, wherein the adjuvant is any compound or composition disclosed herein, and the bacterial infection is thereby treated. In various embodiments the antibiotic or antibacterial agent is a Gram-negative antibiotic or antibacterial agent, a Gram-positive antibiotic or antibacterial agent, or Colistin.

In other various other embodiments, the adjuvant is:

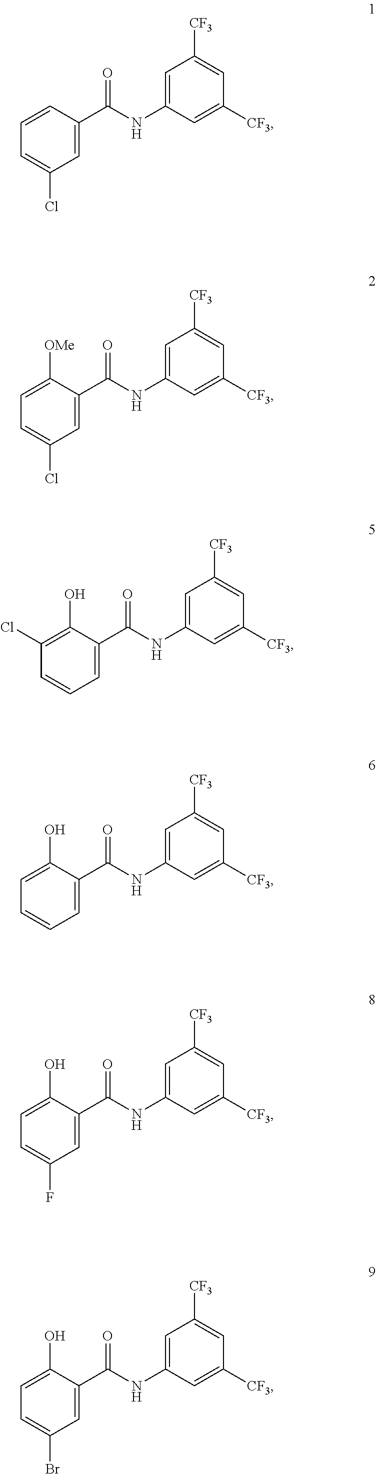 [SMILES: O=C(Nc1cc(C(F)(F)F)cc(C(F)(F)F)c1)c1cccc(Cl)c1 ; COc1ccc(Cl)cc1C(=O)Nc1cc(C(F)(F)F)cc(C(F)(F)F)c1 ; O=C(Nc1cc(C(F)(F)F)cc(C(F)(F)F)c1)c1cccc(Cl)c1O ; O=C(Nc1cc(C(F)(F)F)cc(C(F)(F)F)c1)c1ccccc1O ; O=C(Nc1cc(C(F)(F)F)cc(C(F)(F)F)c1)c1cc(F)ccc1O ; O=C(Nc1cc(C(F)(F)F)cc(C(F)(F)F)c1)c1cc(Br)ccc1O]

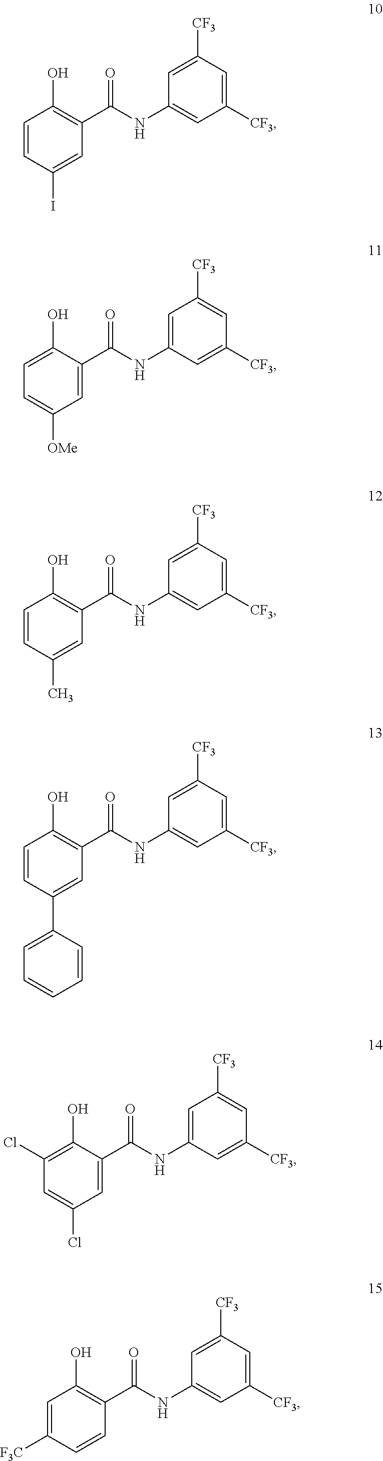 [SMILES: O=C(Nc1cc(C(F)(F)F)cc(C(F)(F)F)c1)c1cc(I)ccc1O ; COc1ccc(O)c(C(=O)Nc2cc(C(F)(F)F)cc(C(F)(F)F)c2)c1 ; Cc1ccc(O)c(C(=O)Nc2cc(C(F)(F)F)cc(C(F)(F)F)c2)c1 ; O=C(Nc1cc(C(F)(F)F)cc(C(F)(F)F)c1)c1cc(-c2ccccc2)ccc1O ; O=C(Nc1cc(C(F)(F)F)cc(C(F)(F)F)c1)c1cc(Cl)cc(Cl)c1O ; O=C(Nc1cc(C(F)(F)F)cc(C(F)(F)F)c1)c1ccc(C(F)(F)F)cc1O]

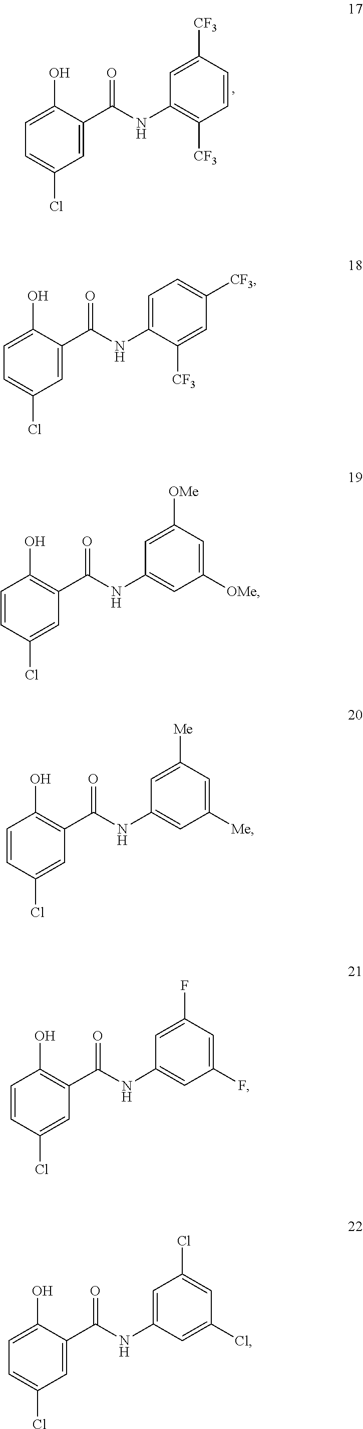 [SMILES: O=C(Nc1cc(C(F)(F)F)ccc1C(F)(F)F)c1cc(Cl)ccc1O ; O=C(Nc1ccc(C(F)(F)F)cc1C(F)(F)F)c1cc(Cl)ccc1O ; COc1cc(NC(=O)c2cc(Cl)ccc2O)cc(OC)c1 ; Cc1cc(C)cc(NC(=O)c2cc(Cl)ccc2O)c1 ; O=C(Nc1cc(F)cc(F)c1)c1cc(Cl)ccc1O ; O=C(Nc1cc(Cl)cc(Cl)c1)c1cc(Cl)ccc1O]

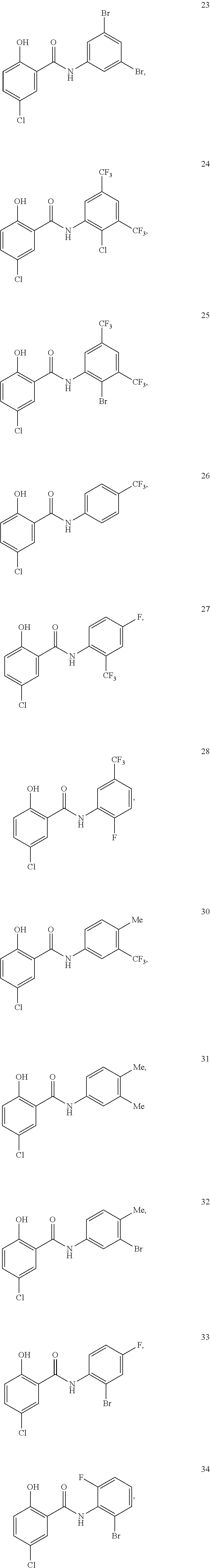

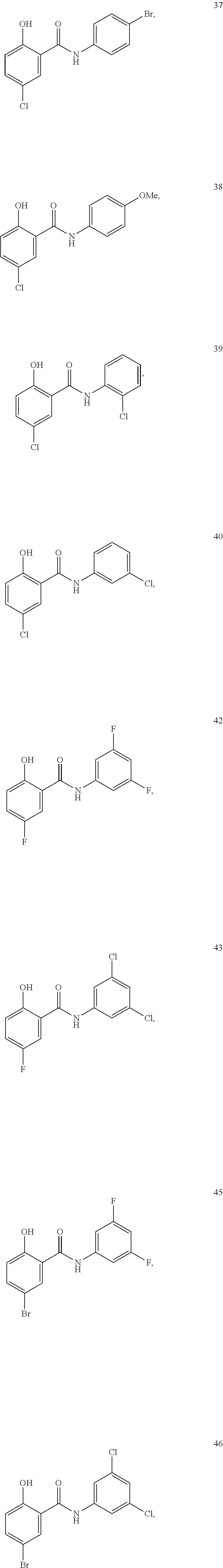

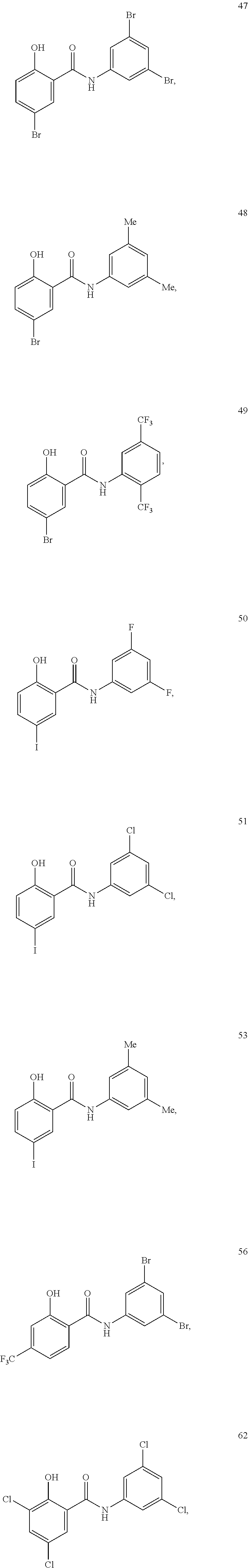

or a pharmaceutical composition thereof.

In some other embodiments, the adjuvant is:

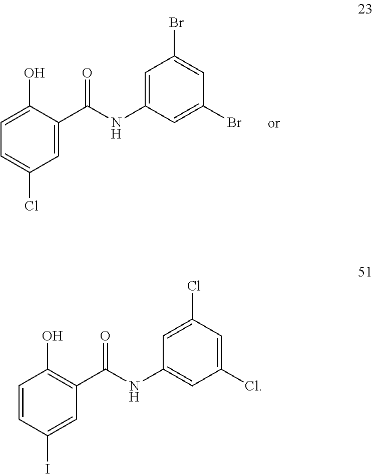 [SMILES: O=C(Nc1cc(Br)cc(Br)c1)c1cc(Cl)ccc1O ; O=C(Nc1cc(Cl)cc(Cl)c1)c1cc(I)ccc1O]

In various additional embodiments, the bacterial infection is a multidrug-resistant Gram-negative bacterial infection. In yet other embodiments, the subject has a systemic concentration of the adjuvant of about 0.01 micromolar to about 10 micromolar. In other embodiments, the adjuvant concentration is about 0.5 micromolar, about 1 micromolar, about 2 micromolar, about 3 micromolar, about 4 micromolar, about 5 micromolar, about 6 micromolar, about 7 micromolar, about 8 micromolar, or about 9 micromolar.

In other embodiments, the subject has a systemic concentration of Colistin of about 0.1 microgram/milliliter to about 50 microgram/milliliter. In other embodiments, the systemic concentration of Colistin is about 0.5 microgram/milliliter, about 1 microgram/milliliter, about 2 microgram/milliliter, about 3 microgram/milliliter, about 4 microgram/milliliter, about 5 microgram/milliliter, about 6 microgram/milliliter, about 7 microgram/milliliter, about 8 microgram/milliliter, about 9 microgram/milliliter, about 10 microgram/milliliter, about 15 microgram/milliliter, about 20 microgram/milliliter, about 25 microgram/milliliter, about 30 microgram/milliliter, about 35 microgram/milliliter, about 40 microgram/milliliter, or about 45 microgram/milliliter.

### Results and Discussion

All IMD-0354 analogues, with the following exceptions were synthesized through a one-step PC₁₃ mediated condensation reaction between a derivatized aniline and corresponding benzoic (salicylic) acid. Compound 2 (Scheme 2) was synthesized by methylating the phenolic hydroxyl group of IMD-0354 with methyl iodide in the presence of potassium carbonate. The bis-phenyl derivative 13 (Scheme 2) was synthesized from the iodo derivative 10 via a Suzuki coupling with phenylboronic acid in the presence of bis(triphenylphosphine)palladium chloride. Finally, aniline 36 was synthesized via a tin (II) chloride mediated reduction of nitro derivative 35 (Scheme 3).

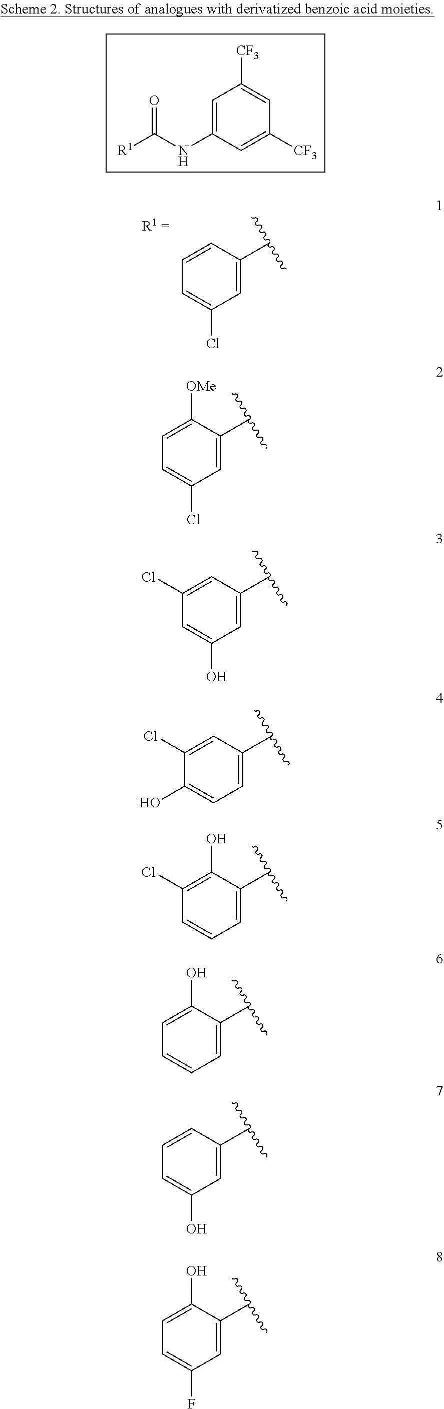

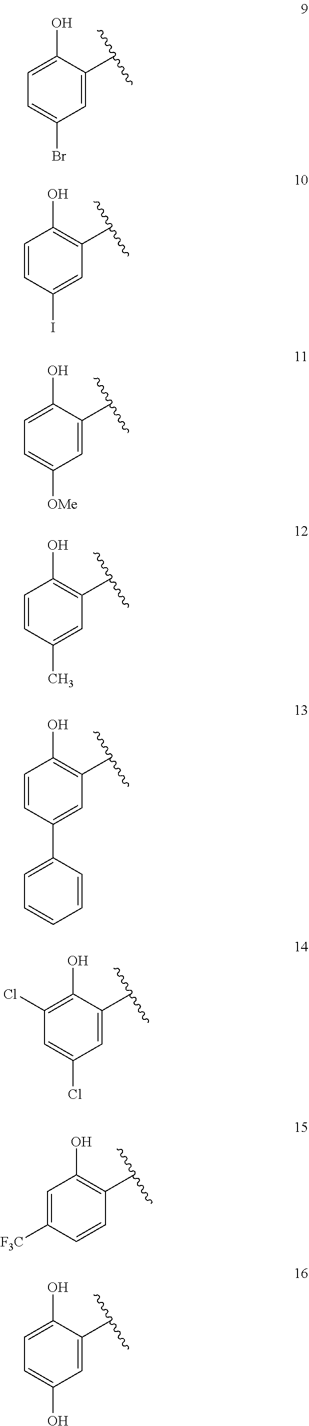

The first compound set we prepared varied the identity of the benzoic acid while retaining the 3,5-bis-trifluoromethylaniline component of IMD-0354 (Scheme 2). All derivatives were screened against two representative, highly colistin-resistant Gram-negative bacterial strains: A. baumannii 4106 (AB4106) and K. pneumoniae B9 (KPB9). In the absence of adjuvant, AB4106 returned a colistin MIC of 2048 μg/mL while KPB9 returned a colistin MIC of 512 μg/mL. The clinical breakpoint for colistin susceptibility against these bacterial species is 2 μg/mL. We have previously established that at 5 μM, IMD-0354 reduces the colistin MIC against AB4106 to 2 μg/mL (1024-fold) while it reduces the colistin MIC against KPB9 to 0.5 μg/mL (1024-fold). To compare directly to IMD-0354, all analogues were thus screened for activity at 5 μM, with any compound displaying an equal or two-fold variation in MIC labeled as equipotent.

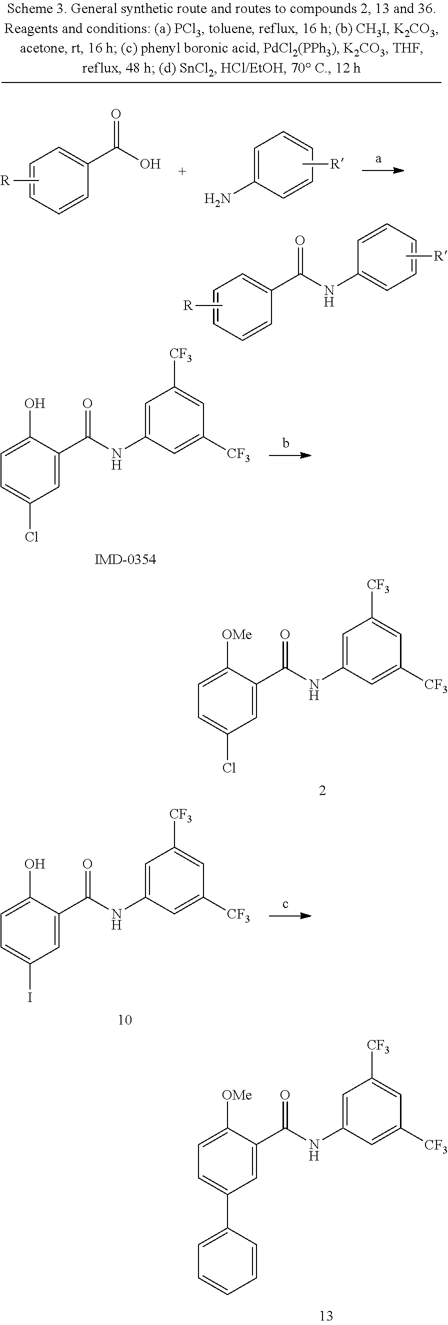

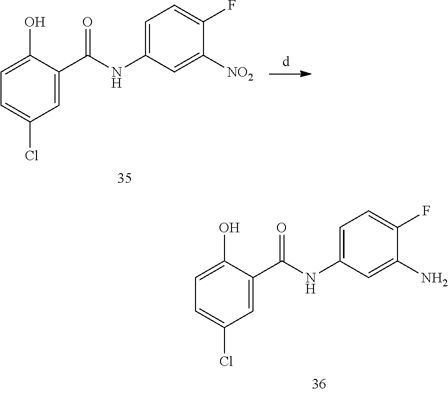 [SMILES: O=C(Nc1ccc(F)c([N+](=O)[O-])c1)c1cc(Cl)ccc1O ; Nc1cc(NC(=O)c2cc(Cl)ccc2O)ccc1F]

The activity of the lead compounds is summarized in Table 1. Full data can be found in Table 4. All new analogues exhibited standalone MICs of greater than or equal to 100 μM, with the exception of compound 60, which exhibited MICs of 50 μM against both strains, compound 25 which exhibited an MIC of 50 μM against AB 4106, compounds 18 and 62, which exhibited an MIC of 50 μM against KP B9, and compounds 9 and 42, which exhibited MICs of 25 μM against KP B9.

We first tested the necessity for the phenolic hydroxyl moiety by either complete removal (1) or methylation (2). Both modifications abrogated activity. Next, activity as a function of position of the phenolic hydroxyl group was probed through compounds 3 and 4. Shifting this hydroxyl group from the ortho to meta (in relationship to the amide, 3) resulted in a compound with essentially equipotent activity to IMD-0354, lowering the colistin MIC to 4 μg/mL against AB4106 and 0.5 μg/mL against KPB9. Further rotation of the hydroxyl group to the para position (4), abolished activity. The impact that the chloro substituent had upon activity was probed by changing its relative position, deletion, or substitution with other halogens (5-10). Moving the position of the chlorine (5), deletion of the chlorine (6), or replacement of the chlorine with either fluorine (8), bromine (9), or iodine (10) was tolerated and delivered compounds with essentially equipotent activity to IMD-0354. Interestingly, the iodo derivative, 10, displayed superior activity to IMD-0354 and reduced the AB4106 colistin MIC to 0.5 μg/mL while it retained equipotent activity against KPB9 (reduced colistin MIC to 0.5 μg/mL). Removal of the chlorine in tandem with moving the phenolic hydroxyl (7) was not tolerated and resulted in a derivative devoid of activity.

TABLE 1
Colistin MICs against AB 4106 and KP B9 in the presence
of lead IMD-0354 analogues.

| | AB 4106 MIC [μg/mL] | KP B9 MIC [μg/mL] |
|---|---|---|
| Compound^{[a]} | (fold reduction) | (fold reduction) |
| IMD-0354 | 2 (1024)^{[b]} | 0.5 (1024) ^{[c]} |
| 9 | 2 (1024) | 0.25 (2048) |
| 10 | 0.5 (4096) | 0.5 (1024) |
| 15 | 1 (2048) | 0.25 (2048) |
| 22 | 2 (1024) | 0.25 (2048) |
| 23 | 2 (1024) | 0.25 (2048) |
| 24 | 0.5 (4096) | 0.5 (1024) |
| 25 | 1 (2048) | 0.25 (2048) |
| 26 | 1 (2048) | 0.25 (2048) |
| 29 | 1 (2048) | 0.5 (1024) |
| 37 | 1 (2048) | 0.25 (2048) |
| 43 | 2 (1024) | 0.125 (4096) |
| 49 | 1 (2048) | 0.25 (2048) |
| 50 | 4 (512) | 0.25 (2048) |
| 51 | 1 (2048) | 0.125 (4096) |
| 52 | 1 (2048) | 0.5 (1024) |
| 55 | 1 (2048) | 0.5 (1024) |
| 56 | 1 (2048) | 0.5 (1024) |
| 60 | 1 (2048) | 0.5 (1024) |

^{[a]}All compounds screened at 5 μM.
^{[b]}MIC of colistin alone is 2048 μg/ mL.
^{[c]}MIC of colistin alone is 512 μg/mL.

We further probed the impact substitution of the chloro group had upon activity by testing the activity of the methoxy analogue 11. This substitution resulted in a significant impact on activity and returned colistin MICs of 8 μg/mL (AB4106) and 16 μg/mL (KPB9). Steric isosteres at the halogen position were constructed for both IMD-0354 (12) and the iodo derivative 10 (analogue 13). The methyl derivative returned similar colistin MICs to IMD-0354 (4 and 0.5 μg/mL against AB4106 and KPB9 respectively), while the phenyl derivative maintained activity against AB4106 (4 μg/mL) but lost some activity against KPB9 (4 μg/mL). The last three derivatives of this subset prepared were the dichloro analogue 14, the trifluoromethyl analogue 15 and the dihydroxylated derivative 16. We found that 15 exhibited equipotent activity to IMD-0354 (1 and 0.25 μg/mL against AB4106 and KPB9 respectively), while compound 14 was four-fold less active than 15 (yet still considered equipotent to IMD-0354). Finally, we noted that 16 was less active against KPB9 (2 μg/mL) and essentially inactive against AB4106. Of the 16 analogues prepared in this subset, we found nine had equipotent or improved activity against both strains in comparison to IMD-0354, while the phenyl derivative had equivalent activity against AB4106.

Next, we probed the structural constraints of the aniline segment of IMD-0354 by fixing the identity of the 5-chlorosalicylic acid and varying the substituents on the aniline ring (Scheme 4). We moved the position of the trifluoromethyl substituents from a 3,5 orientation to a 2,5-(17) and a 2,4-(18) substitution pattern. Both compounds retained activity against KPB9 and AB4106 equivalent to IMD-0354. The requirement for the bis-trifluoromethyl groups was probed by preparing the corresponding 3,5-dimethoxy and 3,5-dimethyl analogues 19 and 20. Although the dimethoxy derivative was bereft of activity, the dimethyl analogue returned colistin MICs of 2 and 1 μg/mL against AB4106 and KPB9 respectively. Previous studies with 2-aminoimidazle-based adjuvants have demonstrated that compounds containing 3,5-dihalogen-ated benzene rings display various activities; therefore, we next studied replacement of the 3,5-bis-trifluoromethyl groups with difluoro, dichloro, and dibromo substituents (21-23). All three analogues were equipotent, suppressing colistin MICs to 2 and 0.25 μg/mL (AB4106 and KPB9). Addition of either a bromine or chlorine at the 2-position of the bis-trifluoroaniline scaffold (24 and 25) again yielded potent compounds returning colistin MICs of 1 and 0.25 μg/mL against AB4106 and KPB9 respectively. Monotrifluoromethyl derivatives 26-30 were then prepared to study the impact retaining one of the trifluoromethyl substituents had upon activity.

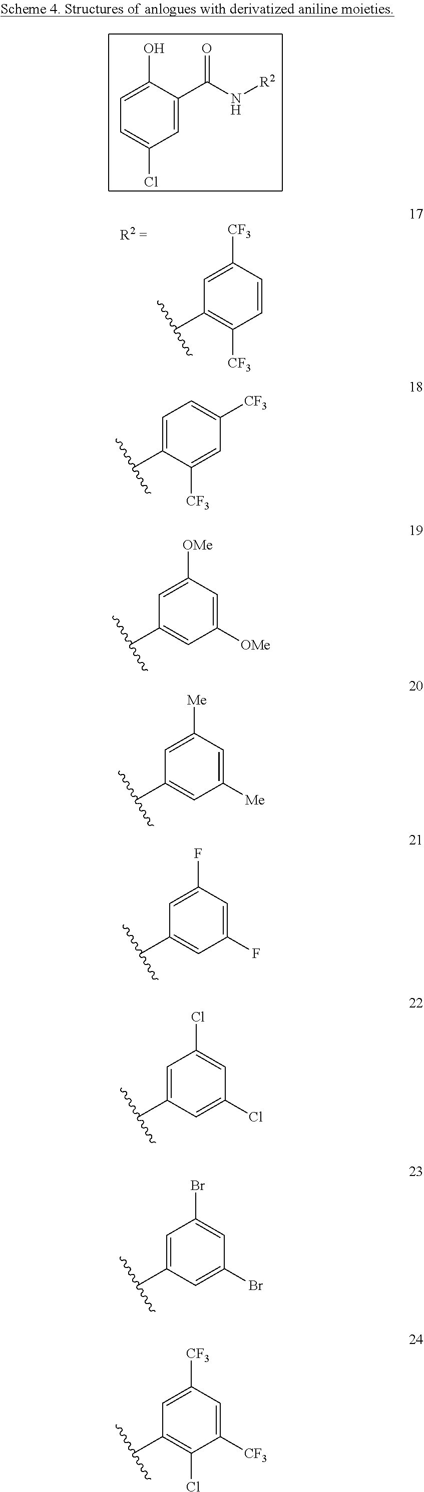

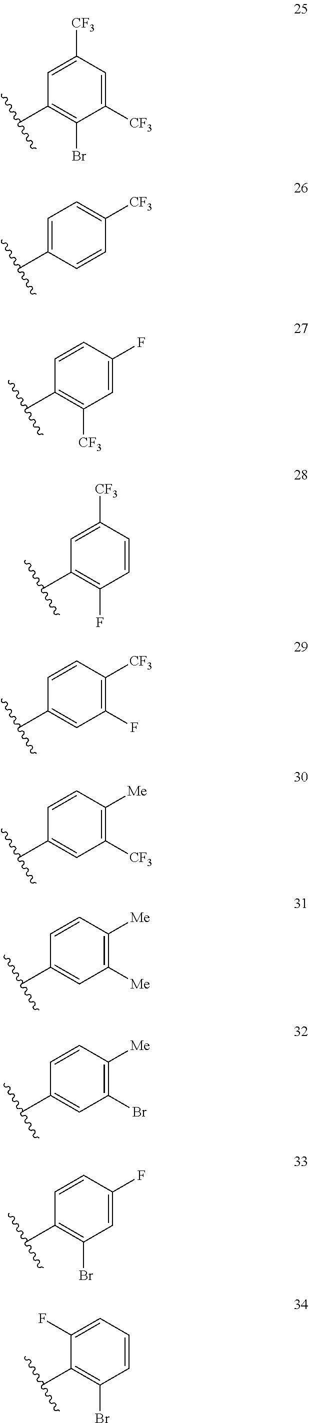

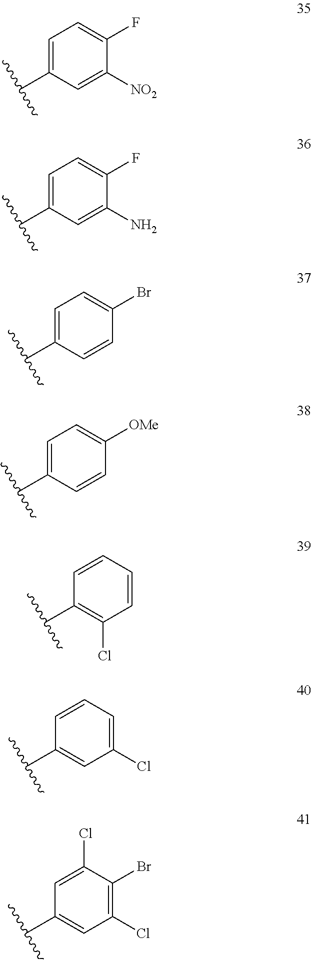

The para-trifluoromethyl derivative 26, as well as the fluorinated analogues 28 and 29, all returned activity equivalent to IMD-0354. Placement of the trifluoromethyl ortho to the amide (27) suppressed activity such that the colistin AB4106 MIC was only reduced to 8 μg/mL while the KPB9 colistin MIC was 1m/mL. Pairing of the trifluoromethyl with a para-methyl was detrimental to activity (30, 64/4 μg/mL against AB4106/KPB9) while the all methyl analogue 31 also showed minimal activity (64/16m/mL against AB4106/KPB9).

Replacement of one of these methyl groups with a bromo substituent, compound 32, only improved activity against KPB9 (8m/mL), while analogues containing an ortho bromo substituent (33 and 34) also showed diminished activity in comparison to IMD-0354 (16/2 and >64/>64 μg/mL against AB4106/KPB9 for 33 and 34 respectively).

We tested further expansion of functional group tolerance on the aniline ring by incorporation of a nitro (35) or an amine (36) substituent in tandem with a para-fluoro substituent. Neither compound displayed significant activity with the exception of 35 against KPB9, which returned an MIC of 1 μg/mL. Next, we focused efforts on monosubstituted aniline building blocks and assembled compounds 37-40. The meta-chloro derivative (40) was active, returning MICs of 2/0.5 μg/mL against AB4106/KPB9, as was the para-bromo derivative 37 (MICs 1/0.25 against AB4106/KPB9), while the ortho-chloro derivative 39 was less active. Mirroring the results from other analogues in this series, incorporation of the electron donating methoxy group abolished activity (38). Finally, we prepared analogue 41 to test the effects of combining the structural components of two active analogues (22 and 37).

This analogue however, showed diminished activity, ca. 4-8 fold over 22 and 37. Overall, we generated 25 derivatives by varying the identity of the aniline substituent, of which 12 showed activity at least equivalent to IMD-0354.

The last compound set we prepared tested the impact of mixing structural elements from both of the previous libraries. The 21 analogues synthesized are depicted in Scheme 5.

With the exception of the 3,5-dimethyl derivatives (44, 48, and 53) and the trifluoro derivative 42, all analogues prepared showed activity essentially equal to IMD-0354 when the halide identity on the salicylic acid building block was varied. Derivatives that contained a 3,5-dihaloaniline building block and either a hydroxyl trifluoromethyl or dichloro salicylic acid fragment were also equally active to IMD-0354 (54-56 and 62), while again all of the corresponding 3,5-dimethyl analogues were less active (57-59). The 2,5-di-trifluoromethyl aniline coupled with the dichloro salicylic acid fragment (61) also returned equipotent activity. Overall, 13 analogues from this last set were identified to have activity levels similar to IMD-0354 with a clear trend that incorporation of a 3,5-dimethyl substitution pattern was detrimental to activity.

Through screening all compounds for activity against AB4106 and KPB9, we identified 37 salicylamide derivatives that we deemed were worthy of further investigation. To further vet activity, we tested whether compound activity was retained against multiple colistin-resistant isolates (Table 2 and Table 5). We chose two additional highly colistin-resistant K. pneumo-niae strains, KPC₃ and KPAS, which have stand-alone colistin MICs of 128 and 2048 μg/mL, respectively, as well as a K. pneumoniae strain harboring the mcr-1 gene (KPF₂₂₁₀₂₉^{mcr 1}, colistin MIC═16 μg/mL).

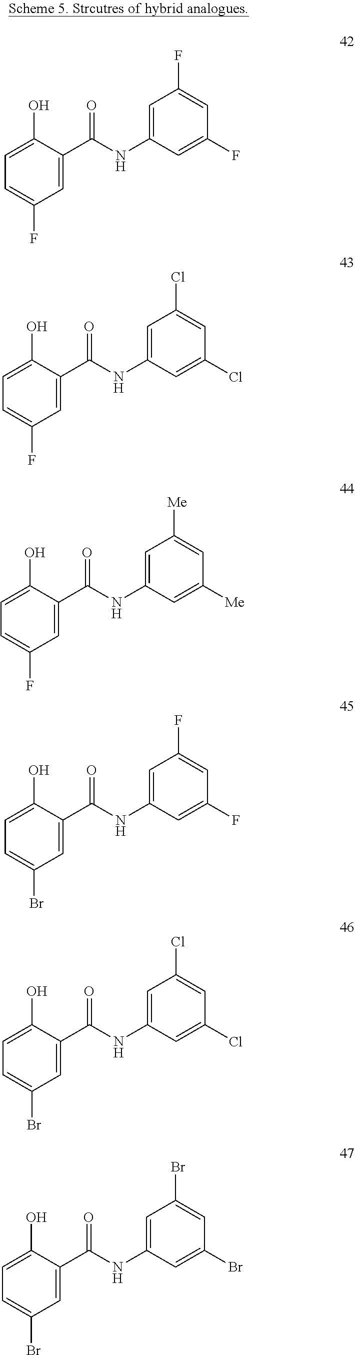 [SMILES: O=C(Nc1cc(F)cc(F)c1)c1cc(F)ccc1O ; O=C(Nc1cc(Cl)cc(Cl)c1)c1cc(F)ccc1O ; Cc1cc(C)cc(NC(=O)c2cc(F)ccc2O)c1 ; O=C(Nc1cc(F)cc(F)c1)c1cc(Br)ccc1O ; O=C(Nc1cc(Cl)cc(Cl)c1)c1cc(Br)ccc1O ; O=C(Nc1cc(Br)cc(Br)c1)c1cc(Br)ccc1O]

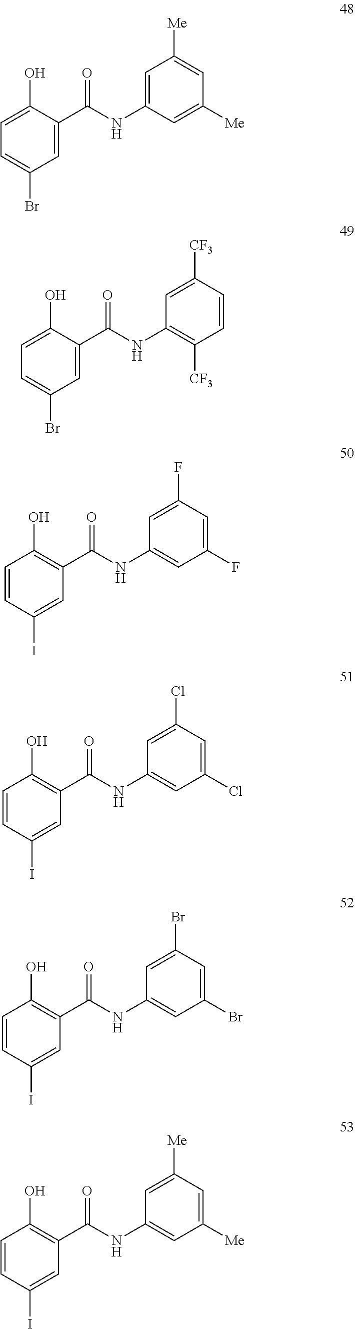 [SMILES: Cc1cc(C)cc(NC(=O)c2cc(Br)ccc2O)c1 ; O=C(Nc1cc(C(F)(F)F)ccc1C(F)(F)F)c1cc(Br)ccc1O ; O=C(Nc1cc(F)cc(F)c1)c1cc(I)ccc1O ; O=C(Nc1cc(Cl)cc(Cl)c1)c1cc(I)ccc1O ; O=C(Nc1cc(Br)cc(Br)c1)c1cc(I)ccc1O ; Cc1cc(C)cc(NC(=O)c2cc(I)ccc2O)c1]

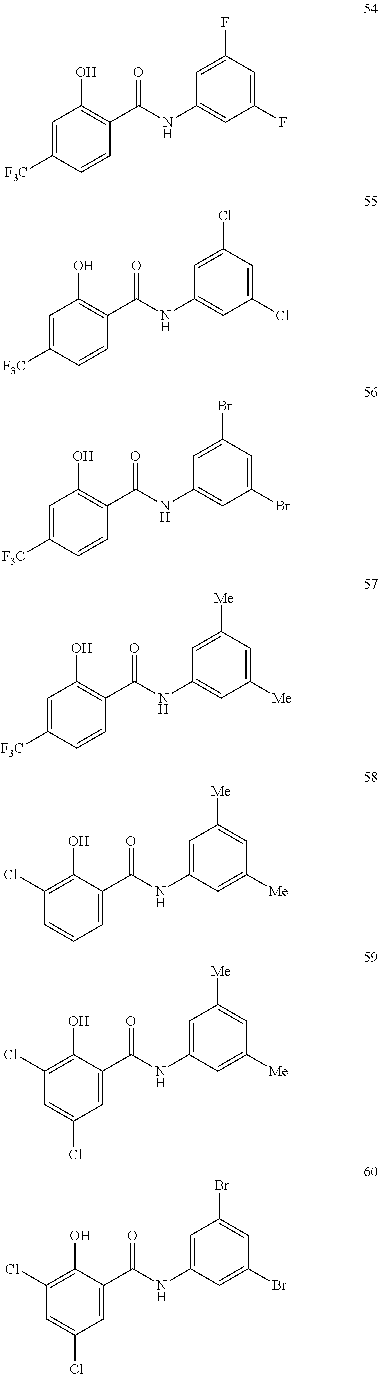

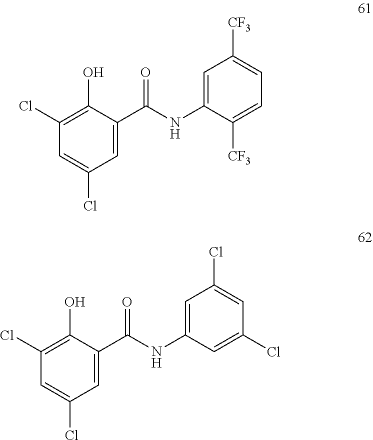 [SMILES: O=C(Nc1cc(C(F)(F)F)ccc1C(F)(F)F)c1cc(Cl)cc(Cl)c1O ; O=C(Nc1cc(Cl)cc(Cl)c1)c1cc(Cl)cc(Cl)c1O]

Compounds were also tested against four additional A. baumannii strains AB3941, AB3942, AB4112, and AB4119, as well as an A. baumannii strain harboring a plasmid containing the mcr-1 gene (AB17978^{mcr 1}). The colistin MICs of these five A. baumannii strains are 1024, >2048, 2048, 1024, and 64 μg/mL respectively. All compounds were screened for their ability to potentiate colistin at 5 μM and compared to IMD-0354 (Table 3).

TABLE 2
Dose-response activity of lead compounds
against KP B9.

| | | Colistin MIC |
|---|---|---|
| | Concentration | [μg/mL] |
| Compound | [μM] | (fold reduction) |
| IMD-0354 | 5 | 0.5 (1024) |
| | 3 | 0.5 (1024) |
| | 1 | 4 (128) |
| 23 | 5 | 0.25 (2048) |
| | 3 | 0.5 (1024) |
| | 1 | 0.5 (1024) |
| | 0.5 | 1 (512) |
| | 0.25 | 4 (128) |
| 29 | 5 | 0.5 (1024) |
| | 3 | 0.5 (1024) |
| | 1 | 0.5 (1024) |
| | 0.5 | 0.5 (1024) |
| | 0.25 | 1 (512) |
| 51 | 5 | 0.125 (4096) |
| | 3 | 0.25 (2048) |
| | 1 | 0.5 (1024) |
| | 0.5 | 1 (512) |
| | 0.25 | 16 (32) |
| 52 | 5 | 0.5 (1024) |
| | 3 | 0.5 (1024) |
| | 1 | 1 (512) |
| | 0.5 | 1 (512) |
| | 0.25 | 16 (32) |

^{a} Colistin MIC in absence of compound is 512 μg/mL

With the exception of KPA5 and AB4119, IMD-0354 suppresses the colistin MIC of all other isolates to at or below the clinical breakpoint level (2 μg/mL). Against KPA5, IMD-0354 suppresses the colistin MIC to 8 μg/mL and against AB4119 it suppresses it to 4 μg/mL. Of the compounds screened, 15 outperformed IMD-0354 and suppressed the colistin MIC against all strains to at or below breakpoint levels.

To further probe activity, we performed a dose—response study with these compounds in comparison to IMD-0354 (Table 2 and Tables 6 and 7) against AB4106 and KPB9. At 3 μM, IMD-0354 suppresses the colistin MIC to 4 μg/mL (AB4106) and 0.5 μg/mL (KPB9), while at 1 μM, it returns colistin MICs of 8 and 4 μg/mL against AB4106 and KPB9. Against AB4106, none of the compounds returned more potent activity than IMD-0354, although 22 compounds performed equally well (MIC within two-fold of that observed with IMD-0354). The most active compounds, 22, 24, 25, 29, and 51 reduce the colistin MIC to 4 μg/mL at 1 μM. Against KPB9, however, 21 analogues outperformed IMD-0354 and returned colistin to at or below breakpoint levels at 1 μM, while 25 derivatives had equipotent activity to IMD-0354. Of the 10 compounds that had superior activity to IMD-0354, four analogues (23, 29, 51 and 52) retained the ability to break colistin resistance at 500 nM, and 29 reduced the colistin MIC below breakpoint levels at 250 nM. All four lead compounds were active against the isolate panel and equally active against AB4106 in comparison to IMD-0354.

TABLE 3
Activity of lead compounds against colistin-resistant isolate panel.

| | Colistin MIC [μg/mL] | | | | | | | |
|---|---|---|---|---|---|---|---|---|
| | (fold reduction) | | | | | | | |
| | KP | KP | KP | AB | AB | AB | AB | AB |
| Cmpd | C3 | A5 | F2210291^{mcr 1} | 3941 | 3942 | 4112 | 4119 | 17978^{mcr 1} |
| | 128 | 2048 | 16 | 1024 | >2048 | 2048 | 1024 | 64 |
| IMD-0354 | 0.5^{[a]} | 8 | 0.5 | 2 | 2 | 2 | 4 | 0.5 |
| | (256) | (256) | (32) | (512) | (≥2048) | (1024) | (256) | (128) |
| 23 | 0.5 | 0.5 | 0.125 | 2 | 2 | 2 | 1 | 0.5 |
| | (256) | (4096) | (128) | (512) | (≥2048) | (1024) | (1024) | (128) |
| 29 | 1 | 1 | 0.25 | 1 | 1 | 1 | 2 | 0.125 |
| | (128) | (2048) | (64) | (1024) | (≥4096) | (2048) | (512) | (512) |
| 51 | 0.25 | 0.25 | 0.25 | 1 | 1 | 1 | 0.5 | 0.125 |
| | (512) | (8192) | (064) | (1024) | (≥4096) | (2048) | (2048) | (512) |
| 52 | 0.5 | 1 | 0.25 | 1 | 1 | 1 | 2 | 0.25 |
| | (256) | (2048) | (64) | (1024) | (≥4096) | (2048) | (512) | (256) |

^{[a]}All compounds tested at 5 μM.

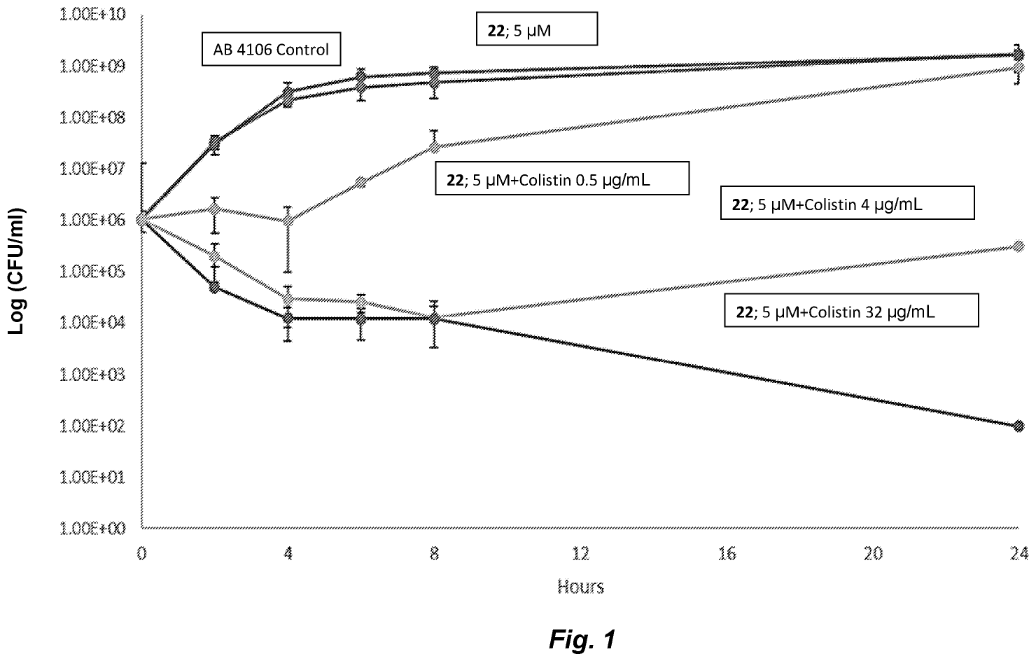

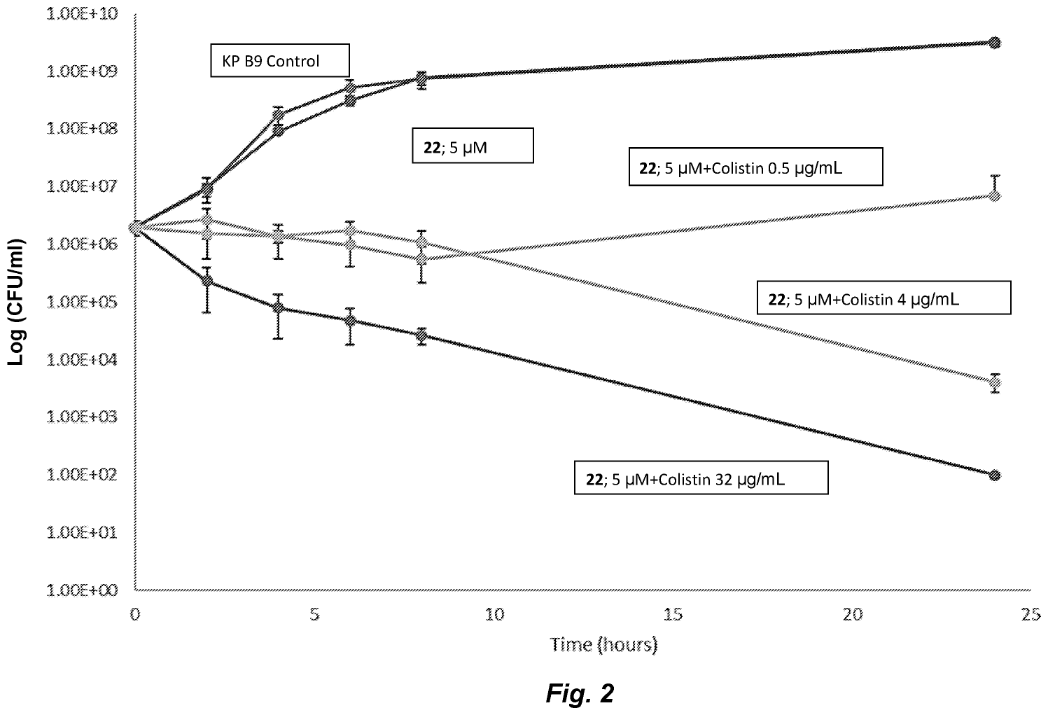

To further quantify activity, and to confirm the non-toxic nature of the adjuvants, we constructed time-kill curves for colistin and compound 22 as a representative lead compound, against both AB4106 and KPB9 (FIGS. 1 and 2). Mirroring the MIC studies, compound 22 showed significant potentiation of colistin activity.

Finally, we tested the cytotoxicity of six compounds (21, 22, 23, 25, 26, and 37) in the absence and presence of 1 μg/mL colistin of the six compounds, 22, 23, 26, and 37 showed no measurable toxicity up to 200 μM. One compound, 21, showed comparable toxicity to IMD-0354 (CT₅₀=210 μM) while another (25) was ca. two-fold more toxic than IMD-0354 (CT₅₀=110 μM). None of the compounds showed additional toxicity in the presence of 1 μg/mL colistin.

In conclusion, we have presented an initial SAR study on the salicylamide lead IMD-0354. A number of structural trends were elucidated, with the most prominent being that the salicylic OH is required for activity (although the position may vary), while the halogen identity is flexible. On the aniline fragment, the trifluoromethyl groups can be exchanged with halogens, while electron donating groups are generally not tolerated. Through these studies, we have managed to identify a number of analogues with activity similar to or exceeding IMD-0354. Four of these leads (23, 29, 51, and 52) show activity in the nanomolar range, which marks them as some of the most potent colistin adjuvants identified to date. As a potential treatment for colistin-resistant infections, IMD-0354 is hampered by solubility and its inhibition of inflammation through targeting IKK-β. To this end, we are currently conducting additional SAR studies on this scaffold in efforts to develop analogues that retain their colistin adjuvant activity, yet are more soluble in aqueous media with muted effect on IKK-β.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet.

Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-bacterial agents and have higher potency and/or reduced toxicity as compared to IMD-0354. Preferably, compounds of the invention are more potent and less toxic than IMD-0354, and/or avoid a potential site of catabolic metabolism encountered with IMD-0354, i.e., have a different metabolic profile than IMD-0354.

The invention provides therapeutic methods of treating infections in a mammal, which involve administering to a mammal having an infection an effective amount of a compound or composition described herein. A mammal includes a primate, human, rodent, canine, feline, bovine, ovine, equine, swine, caprine, bovine and the like.

The ability of a compound of the invention to treat infections may be determined by using assays well known to the art. For example, the design of treatment protocols, toxicity evaluation, data analysis, and quantification of bacterium kill, are known. In addition, ability of a compound to treat infections may be determined using the tests as described below.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### General Chemistry Experimental and Compound Characterization

All reactions were carried out under an atmosphere of nitrogen using anhydrous solvents unless otherwise specified. All chemical reagents for synthesis were used without further purification. Analytical thin layer chromatography (TLC) was performed using 250 μm Silica Gel 60 F₂₅₄ pre-coated plates (EMD Chemicals Inc.). Flash column chromatography was performed using 230-400 Mesh 60 A Silica Gel from Sorbent Technologies. NMR spectra were recorded using broadband probes on a Bruker AVANCE III HD 400 Nanobay (400 MHz for 1H and 100 MHz for 13C). All spectra are presented using MestReNova (Mnova) software and 1H NMR are typically displayed from 12 to -0.7 ppm without the use of the signal suppression function. Spectra were obtained in the following solvents (reference peaks also included for 1H and 13C NMRs): CDC₁₃ (1H NMR: 7.26 ppm; 13C NMR: 77.23 ppm), d6-DMSO (1H NMR: 2.50 ppm; 13C NMR: 39.52 ppm) and d4-MeOD (1H NMR: 3.31 ppm; 13C NMR: 49.00 ppm). All NMR experiments were performed at room temperature. Chemical shift values (6) are reported in parts per million (ppm) for all 1H NMR and 13C NMR spectra. 1H NMR multiplicities are reported as: s=singlet, d=doublet, t=triplet, q=quartet, p=pentet, m=multiplet, br=broad. High-resolution mass spectra were obtained for all new compounds from the mass spectrometry and proteomics facility at university of Notre Dame performed on a Bruker-TOF-ESI spectrometer in positive module using direct infusion in 9:1 acetonitrile: water. IR spectra were recorded on Bruker Alpha II FTIR spectrometer. UV data was taken using a Thermo Scientific, Genesys 10 UV scanning spectrometer.

General procedure for synthesis of N-aryl-2-hydroxybenzamides: To a stirring solution of salicylic acid (100mg, 1 eq) in toluene under nitrogen atmosphere, was added phosphorus trichloride (0.5 eq) dropwise and heated to reflux. Substituted aniline (0.9 eq) was added in portions over 10 minutes and the reaction mixture was refluxed overnight. The reaction was checked for completion by TLC, then cooled and ethyl acetate (100 mL) added. The suspension was washed with 2N HC₁ (3×30 mL) followed by brine (30 mL) and then aqueous sodium bicarbonate (3×30 mL). The organic layer was washed with brine (30 mL) then dried using anhydrous sodium sulfate, evaporated and purified via flash chromatography using 1:2 to 1:1 DCM/hexanes to obtain a white solid. Compounds were tested without further purification from stock solutions in biological grade DMSO.

N-(3,5-bis(trifluoromethyl)phenyl)-3-chloro-5-hydroxybenzamide (3): The title compound was synthesized following the general procedure for synthesis of N-aryl-2-hydroxybenzamides to afford 3 as a white solid (52 mg, 39%). 1H NMR (400 MHz, d3-Acetonitrile): 9.11 (s, 1H), 8.41−8.30 (m, 2H), 7.75 (s, 1H), 7.46 (t, J=1.4, 0.7 Hz, 1H), 7.30 (t, J=1.6 Hz, 1H), 7.08 (t, J=2.0 Hz, 1H). 13C NMR (100 MHz, d3-Acetonitrile): 164.5, 157.8, 140.1, 136.6, 134.3, 131.1 (q, J=33.3 Hz), 123.1 (q, J=271.8 Hz), 119.8, 119.8, 118.7, 118.5, 113.1. UV (λmax nm): 265; IRvmax (cm-1): 3324, 1654, 1573, 914; HRMS (ESI): calcd for C15H₉ClF6NO2[M+H]+: 384.0221, found: 384.0213.

N-(3,5-bis(trifluoromethyl)phenyl)-3-chloro-4-hydroxybenzamide (4): The title compound was synthesized following the general procedure for synthesis of N-aryl-2-hydroxybenzamides to afford 4 as a white solid (76 mg, 14%). 1H NMR (400 MHz, d6-DMSO): 11.11 (s, 1H), 10.66 (s, 1H), 8.50 (s, 2H), 8.09 (d, J=2.3 Hz, 1H), 7.84 (dd, J=8.6, 2.3 Hz, 1H), 7.80 (s, 1H), 7.11 (d, J=8.5 Hz, 1H). 13C NMR (100 MHz, d6-DMSO): 164.6, 156.8, 141.2, 130.7 (q, J=32.7 Hz), 129.7, 128.6, 125.3, 123.4 (q, J=272.8 Hz), 119.8 (d, J=4.2 Hz), 119.7, 116.4, 116.2 (m) . UV (λmax nm): 272; IRvmax (cm-1): 3174, 1643, 1118, 681; HRMS (ESI): calcd for C₁₅H₉C₁F₆NO2[M+H]+: 384.0221, found: 384.0194.

N-(3,5-bis(trifluoromethyl)phenyl)-3-hydroxybenzamide (7): The title compound was synthesized following the general procedure for synthesis of N-aryl-2-hydroxybenzamides to afford 7 as a white solid (34 mg, 23%). 1H NMR (400 MHz, d6-DMSO): 10.76 (s, 1H), 9.86 (s, 1H), 8.53 (d, J=1.8 Hz, 2H), 7.81 (s, 1H), 7.43 (dt, J=7.6, 1.3 Hz, 1H), 7.40−7.33 (m, 2H), 7.03 (ddd, J=7.9, 2.4, 1.1 Hz, 1H). 13C NMR (100 MHz, d6-DMSO): 13C NMR (100 MHz, d6-DMSO): 166.3, 157.5, 141.2, 135.3, 130.6 (q, J=32.8 Hz), 129.7, 123.3 (d, J=272.6 Hz), 119.8 (q, J=3.4 Hz), 119.3, 118.3, 116.4 (q, J=7.3 Hz), 114.6. UV (λmax nm): 274; IR vmax (cm-1): 3080, 1564, 1509, 1220; HRMS (ESI): calcd for C14H13ClNO3 [M+H]+: 278.0578, found: 278.0596.

N-(3,5-bis(trifluoromethyl)phenyl)-2,5-dihydroxybenzamide (16): The title compound was synthesized following the general procedure for synthesis of N-aryl-2-hydroxybenzamides to afford 16 as a white solid (70 mg, 26%). 1H NMR (400 MHz, d6-DMSO): 11.04 (s, 1H), 8.41 (s, 1H), 7.82 (s, 1H), 7.17 (t, J=8.2 Hz, 1H), 6.43 (d, J=8.2 Hz, 2H). 13C NMR (100 MHz, d6-DMSO): 167.8, 158.3, 140.3, 132.9, 130.8 (q, J=32.8 Hz), 123.3 (q, J=272.8 Hz), 120.4, 120.4, 116.8, (q, J=4.2 Hz), 116.7, 107.5, 107.1. UV (λmax nm): 275; IRvmax (cm-1): 3325, 1644, 1445, 974. HRMS (ESI): calcd for C15H10F6NO3 [M+H]+: 366.0559, found: 366.0541.

5-chloro-N-(3-fluoro-4-(trifluoromethyl)phenyl)-2-hydroxybenzamide (29): The title compound was synthesized following the general procedure for synthesis of N-aryl-2-hydroxybenzamides to afford 29 as a white solid (21 mg, 9%). 1H NMR (400 MHz, d6-DMSO): 11.44 (s, 1H), 10.86−10.81 (m, 1H), 7.96 (d, J=13.4 Hz, 1H), 7.79 (dd, J=15.3, 5.8 Hz, 2H), 7.67 (d, J=8.8 Hz, 1H), 7.48 (d, J=8.8 Hz, 1H), 7.04 (d, J=8.8 Hz, 1H) . 13C NMR (100 MHz, d6-DMSO): 165.1, 159.1 (d, J=249.5 Hz), 155.9, 144.2 (d, J=11.3 Hz), 133.1, 128.8, 127.9 (qd, J=6.8, 4.5 Hz),122.9, 122.8 (q, J=271.0 Hz), 120.8, 119.0, 115.9 (d, J=3.1 Hz), 111.3 (dd, J=32.7, 12.5 Hz), 107.8 (d, J=25.4 Hz). UV (λmax nm): 268; IRvmax (cm-1): 2924, 1606, 1122, 695; HRMS (ESI): calcd for C14H9C1F₄NO₂ [M+H]+:334.0252, found: 334.0232.

5-chloro-N-(4-fluoro-3-nitrophenyl)-2-hydroxybenzamide (35): The title compound was synthesized following the general procedure for synthesis of N-aryl-2-hydroxybenzamides to afford 35 as a white solid (67 mg, 45%). 1H NMR (400 MHz, d6-DMSO): 11.51 (s, 1H), 10.76 (s, 1H), 8.66 (dd, J=6.9, 2.7 Hz, 1H), 8.05 (dt, J=9.2, 3.3 Hz, 1H), 7.87 (d, J=2.7 Hz, 1H), 7.62 (dd, J=11.2, 9.1 Hz, 1H), 7.48 (dd, J=8.8, 2.7 Hz, 1H), 7.03 (d, J=8.8 Hz, 1H). 13C NMR (100 MHz, d6-DMSO): 165.2, 156.4, 150.9 (d, J=259.3 Hz), 136.4 (d, J=7.6 Hz), 135.0 (d, J=3.4 Hz), 133.1, 128.5, 128.0 (d, J=8.3 Hz), 122.7, 120.2, 119.1, 118.9 (d, J=21.8 Hz), 117.1 (d, J=3.0 Hz). UV (λmax nm): 254; IRvmax (cm-1): 3291, 1620, 1334, 932; HRMS (ESI): calcd for C13H9C1FN2O4 [M+H]+: 311.0229, found: 311.0241.

N-(3-amino-4-fluorophenyl)-5-chloro-2-hydroxybenzamide (36): To a solution of 5-chloro-N-(4-fluoro-3-nitrophenyl)-2-hydroxybenzamide 35 (1 eq) in anhydrous ethanol (0.1 M) was added tin (II) chloride (2 eq). The reaction mixture was heated to 70° C. for 12 h, cooled, and concentrated under reduced pressure. The residue was taken in a mixture of 100 mL ethyl acetate and 100 mL saturated aqueous sodium bicarbonate solution and the organic layer was washed with brine, dried over anhydrous sodium sulfate, filtered, and concentrated under reduced pressure, and then purified via flash chromatography using 10:1 DCM/methanol to obtain compound 36 as a white solid (112 mg, 71%). 1H NMR (400 MHz, d6-DMSO): 11.94 (s, 1H), 10.22 (s, 1H), 7.95 (d, J=2.7 Hz, 1H), 7.45 (dd, J=8.8, 2.7 Hz, 1H), 7.20 (dd, J=8.4, 2.6 Hz, 1H), 7.00 (d, J=9.0 Hz, 1H), 6.97−6.91 (m, 1H), 6.77 (ddd, J=8.7, 4.1, 2.6 Hz, 1H), 5.24 (s, 2H). 13C NMR (100 MHz, d6-DMSO): 165.2, 157.5, 148.0 (d, J=234.7 Hz), 136.9 (d, J=13.7 Hz), 134.8 (d, J=2.3 Hz), 133.4, 128.7, 123.1, 119.9, 119.6, 115.1 (d, J=19.3 Hz), 109.3 (d, J=4.4 Hz), 108.9 (d, J=6.5 Hz). UV (λmax nm): 274; IRvmax (cm-1): 3351, 1651, 1128, 933; HRMS (ESI): calcd for C₂₁H₁₄F₆NO₂ [M+H]+: 281.04876, found: 281.0488.

N-(4-bromo-3,5-dichlorophenyl)-5-chloro-2-hydroxybenzamide (41): The title compound was following the general procedure for synthesis of N-aryl-2-hydroxybenzamides to afford 41 as a white solid (53 mg, 19%). 1H NMR (400 MHz, d6-DMSO): 11.43 (s, 1H), 10.60 (s, 1H), 8.03 (s, 2H), 7.81 (d, J=2.7 Hz, 1H), 7.48 (dd, J=8.8, 2.7 Hz, 1H), 7.03 (d, J=8.8 Hz, 1H) . 13C NMR (100 MHz, d6-DMSO): 165.1, 156.0, 139.1, 135.1, 133.2, 128.7, 122.8, 120.5, 120.2, 119.0, 116.1. UV(kmax nm): 248; IRvmax (cm-1): 3149, 1626, 1208, 655; HRMS (ESI): calcd for C13H8BrC13NO2[M+H]+: 393.8799, found: 393.8808. N-(3,5-dimethylphenyl)-5-fluoro-2-hydroxybenzamide (44): The title compound was synthesized following the general procedure for synthesis of N-aryl-2-hydroxybenzamides to afford 44 as a white solid (58 mg, 43%). 1H NMR (400 MHz, d6-DMSO): 11.7 (s, 1H), 10.3 (s, 1H), 7.8 (dd, J=9.8, 3.2 Hz, 1H), 7.3 (d, J=1.4 Hz, 2H), 7.3-7.3 (m, 1H), 7.0 (dd, J=9.0, 4.6 Hz, 1H), 6.8 (s, 1H), 2.3 (s, 6H). 13C NMR (100 MHz, d6-DMSO): 165.3 (d, J=2.5 Hz), 155.3 (d, J=235.0 Hz), 154.9, 138.3 (d, J=2.8 Hz), 126.3, 121.0, 120.8, 119.1 (d, J=7.6 Hz), 119.0, 118.9 (d, J=6.9 Hz), 115.2 (d, J=24.4 Hz), 21.5. UV (λmax nm): 271; IRvmax (cm-1): 3086, 1634, 1578, 912; HRMS (ESI): calcd for C15H15FNO2 [M+H]+: 260.1081, found: 260.1064.

N-(3,5-dibromophenyl)-2-hydroxy-5-iodobenzamide (52): The title compound was synthesized following the general procedure for synthesis of N-aryl-2-hydroxybenzamides to afford 52 as a white solid (53 mg, 13%). 1H NMR (400 MHz, d6-DMSO): 11.46 (s, 1H), 10.59 (s, 1H), 8.08 (d, J=2.3 Hz, 1H), 8.00 (d, J=1.7 Hz, 2H), 7.71 (dd, J=8.7, 2.3 Hz, 1H), 7.59 (t, J=1.7 Hz, 1H), 6.83 (d, J=8.6 Hz, 1H) . 13C NMR (100 MHz, d6-DMSO): 165.1, 158.6, 141.5, 141.2, 137.2, 128.4, 122.3, 121.7, 120.3, 79.9. UV (λmax nm): 236; IRvmax (cm-1): 3079, 1636, 1109, 664; HRMS (ESI): calcd for C13H9Br2INO2[M+H]+: 495.8039, found: 495.8059.

N-(3,5-difluorophenyl)-2-hydroxy-4-(trifluoromethyl)benzamide (54): The title compound was synthesized following the general procedure for synthesis of N-aryl-2-hydroxybenzamides to afford 54 as a white solid (191 mg, 50%).1H NMR (400 MHz, d6-DMSO): 11.54 (s, 1H), 10.70 (s, 1H), 7.88 (d, J=7.9 Hz, 1H), 7.53−7.45 (m, 2H), 7.29 (d, J=8.5 Hz, 2H), 7.00 (tt, J=9.4, 2.4 Hz, 1H) . 13C NMR (100 MHz, d6-DMSO): 164.9, 162.5 (dd, J=243.2, 15.3 Hz), 156.5, 141.0 (t, J=13.7 Hz), 132.5 (q, J=31.8 Hz), 130.8, 124.5, 123.6 (q, J=272.7 Hz), 115.5 (q, J=3.8 Hz), 113.3 (q, J=3.9 Hz), 103.0 (dd, J=20.8, 8.9 Hz), 99.2 (t, J=26.2 Hz) . UV (λmax nm): 268; IRvmax (cm-1): 3173, 1620, 1113, 841;

HRMS (ESI): calcd for C14H9F5NO2 [M+H]+: 318.0548, found: 318.0536.

N-(3,5-dichlorophenyl)-2-hydroxy-4-(trifluoromethyl)benzamide (55): The title compound was synthesized following the general procedure for synthesis of N-aryl-2-hydroxybenzamides to afford 55 as a white solid (239 mg, 56%). 1H NMR (400 MHz, d6-DMSO): 11.54 (s, 1H), 10.65 (s, 1H), 7.89 (d, J=7.9 Hz, 1H), 7.84 (d, J=1.9 Hz, 2H), 7.37 (t, J=2.0 Hz, 1H), 7.29 (d, J=8.7 Hz, 2H) . 13C NMR (100 MHz, d6-DMSO): 165.0, 156.6, 140.8, 134.1, 132.6 (q, J=31.9 Hz), 130.8, 124.3, 123.6 (q, J=272.8 Hz), 123.3, 118.3, 115.5 (q, J=3.7 Hz), 113.3 (q, J=4.0 Hz). UV (λmax nm): 270; IRvmax (cm-1): 3081, 1640, 1110, 664; HRMS (ESI): calcd for C₁₄H₉C₁₂F₃NO_{2 [}M+H]+: 349.9957, found: 349.9935.

N-(3,5-dimethylphenyl)-2-hydroxy-4-(trifluoromethyl)benzamide (57): The title compound was synthesized following the general procedure for synthesis of N-aryl-2-hydroxybenzamides to afford 57 as a white solid (25 mg, 14%). 1H NMR (400 MHz, d6-DMSO): 11.91 (s, 1H), 10.32 (s, 1H), 8.02 (d, J=8.0 Hz, 1H), 7.34 (s, 2H), 7.29 (d, J=9.3 Hz, 2H), 6.79 (s, 1H), 2.27 (s, 6H) . 13C NMR (100 MHz, d6-DMSO): 164.6, 157.4, 138.1, 137.9, 132.6 (q, J=32.0 Hz), 130.7, 125.9, 123.6 (q, J=272.8 Hz), 123.4, 118.3, 115.4 (q, J =3.6 Hz), 113.6 (q, J=3.8 Hz), 21.1 . UV (λmax nm): 302; IRvmax (cm-1): 2588, 1620, 1119, 681; HRMS (ESI): calcd for C16H₁₅F₃NO_{2 [}M+H]+: 310.1049, found: 310.1046.

3-chloro-N-(3,5-dimethylphenyl)-2-hydroxybenzamide (58): The title compound was synthesized following the general procedure for synthesis of N-aryl-2-hydroxybenzamides to afford 58 as a white solid (45 mg, 27%). 1H NMR (400 MHz, d6-DMSO): 12.9 (s, 1H), 10.5 (s, 1H), 8.0 (dd, J=8.1, 1.5 Hz, 1H), 7.7 (dd, J=7.9, 1.4 Hz, 1H), 7.3 (s, 2H), 7.0 (t, J=8.0 Hz, 1H), 6.8 (s, 1H), 2.3 (s, 6H). 13C NMR (100 MHz, d6-DMSO): 167.8, 156.2, 137.8, 137.2, 134.0, 126.8, 126.4, 121.3, 119.6, 119.1, 117.2, 21.0. UV (λmax nm): 271; IRvmax (cm-1): 3380, 1614, 1549, 1117; HRMS (ESI): calcd for C15H₁₅C₁NO_{2 [}M+H]+: 276.0786, found: 276.0767.

3,5-dichloro-N-(3,5-dimethylphenyl)-2-hydroxybenzamide (59): The title compound was synthesized following the general procedure for synthesis of N-aryl-2-hydroxybenzamides to afford 59 as a white solid (65 mg, 44%). 1H NMR (400 MHz, d6-DMSO): 12.9 (s, 1H), 10.7 (s, 1H), 8.1 (t, J=2.9, 1.5 Hz, 1H), 7.8 (d, J=2.6 Hz, 1H), 7.3 (s, 2H), 6.8 (s, 1H), 2.3 (s, 6H). 13C NMR (100 MHz, d6-DMSO): 166.4, 155.5, 137.8, 137.2, 133.0, 126.4, 122.7, 121.8, 119.3, 118.3, 118.2, 21.0. UV (λmax nm): 265; IRvmax (cm-1): 3234, 1632, 1572, 1168. HRMS (ESI): calcd for C15H₁₄C₁₂NO_{2 [}M+H]+: 310.0396, found: 310.0385.

3,5-dichloro-N-(3,5-dibromophenyl)-2-hydroxybenzamide (60): The title compound was synthesized following the general procedure for synthesis of N-aryl-2-hydroxybenzamides to afford 60 as a white solid (25 mg, 19%). 1H NMR (400 MHz, d6-DMSO): 14.75 (s, 1H), 7.92 (d, J=1.7 Hz, 2H), 7.64 (d, J=3.0 Hz, 1H), 7.44 (t, J=1.7 Hz, 1H), 7.34 (d, J=3.0 Hz, 1H). 13C NMR (100 MHz, d6-DMSO): 166.3, 163.9, 143.0, 131.7, 127.5, 127.4, 126.1, 122.9, 121.3, 119.3, 113.3. UV (λmax nm): 278; IRvmax (cm-1): 3293, 1640, 1579, 145; HRMS (ESI): calcd for C13H8Br2C12NO2 [M+H]+: 437.8293, found: 437.8261.

N-(2,5-bis(trifluoromethyl)phenyl)-3,5-dichloro-2-hydroxybenzamide (61): The title compound was synthesized following the general procedure for synthesis of N-aryl-2-hydroxybenzamides to afford 61 as a white solid (21 mg, 12%). 1H NMR (400 MHz, d3-Acetonitrile): 9.0 (s, 1H), 7.8 (d, J=8.3 Hz, 1H), 7.7 (d, J=3.0 Hz, 1H), 7.5 — 7.4 (m, 1H), 7.3 (d, J=3.0 Hz, 1H). 13C NMR (100 MHz, d3-Acetonitrile): 167.6, 166.4, 140.3, 134.5, 134.2, 132.3, 128.7, 128.0 (q, J=5.8 Hz), 127.2, 124.8 (q, J=271.9 Hz), 124.6 (q, J=271.9 Hz), 121.8 (q, J=4.1 Hz), 119.9, 119.4 (q, J=3.8 Hz), 113.3. UV (λmax nm): 273; IRvmax (cm-1): 3291, 1640, 1542, 1120; HRMS (ESI): calcd for C15H₇C₁₂F₆NO₂Na [M+Na]+: 439.9650, found: 439.9659.

### Example 2

### General Biological Experimental

Bacterial strains, media, and antibiotics: A. baumannii strains 4106, 4112, 4119, 3941 and 3942 were obtained from Walter Reed Army Institute for Research (WRAIR). A. baumannii strain 17978mcr 1 and K. pneumoniae strains B9, A5, C₃ and F₂₂₁₀₂₉₁mcr 1 were obtained from Professor Robert Ernst at The University of Maryland, Baltimore. Stock cultures were stored in 25% glycerol and maintained at 80 C. Prior to use, colonies were grown on LB (Lennox) agar. CAMHB was purchased from BD Diagnostics. Colistin sulfate salt was purchased from Sigma Aldrich (Cat# C₄₄₆₁). All assays were run in duplicate and repeated at least two separate times.

Broth microdilution method for the determination of minimum inhibitory concentrations: Bacteria were cultured for 4 to 6 hours in CAMHB and subcultured to 1.04×106 CFU/mL for AB 4106 and 1.96×106 CFU/mL for KP B9 in fresh CAMHB. To aliquots (1 mL) was added compound from stock solutions in DMSO, such that the compound concentration equaled the highest concentration tested. Samples were then dispensed (200 μL) into the first row of a 96-well microtiter plate in which all but the final row of subsequent wells were prefilled with 100 μL of the untreated bacterial subculture. The final row was filled with media to act as a sterility control and blank. Row one wells were mixed 6-7 times, then, 100 μL was withdrawn and transferred to row two. Row two wells were mixed 6-7 times followed by a 100 μL transfer from row two to row three. This procedure was used to serially dilute the rest of the rows of the microtiter plate, excluding the last prefilled row, which was used to measure growth in the absence of compound. Plates were then sealed with GLAD Press'n Seal and incubated under stationary conditions at 37 C. After 16 hours, the plates were removed, and MIC values were measured by recording the OD600 of each well. MIC values were determined as the minimum concentration required to achieve 90% growth inhibition compared to growth in untreated wells.

Broth microdilution method for measurement of colistin potentiation: Bacteria were cultured for 4 to 6 hours in CAMHB and diluted to 1.04×106 CFU/mL for AB 4106 and 1.96×106 CFU/mL for KP B9 in fresh CAMHB. To aliquots (4 mL) was added compound from stock solutions in DMSO. One aliquot was not dosed to allow measurement of the colistin MIC in the absence of compound. A 1 mL aliquot of each sample was dosed with colistin, and from this 200 μL was dispensed into the first row of a 96-well microtiter plate in which all but the final row of subsequent wells was prefilled with 100 μL of the corresponding compound dosed bacterial suspension The final row was filled with media to act as a sterility control and blank. Row one wells were mixed 6-7 times, then, 100 μL was withdrawn and transferred to row two. Row two wells were mixed 6-7 times followed by a 100 μL transfer from row two to row three. This procedure was used to serially dilute the rest of the rows of the microtiter plate, excluding the last prefilled row, which was used to measure growth in the presence of compound alone. Plates were then sealed with GLAD

Press'n Seal and incubated under stationary conditions at 37° C. After 16 hours, the plates were removed, and MIC values were measured by recording the OD600 of each well. MIC values were determined as the minimum concentration required to achieve 90% growth inhibition compared to growth in untreated wells.

Time—kill Curves: Strains were cultured overnight in CAMHB and subcultured to 1.04×106 CFU/mL for AB 4106 and 1.96×106 CFU/mL for KP B9 in fresh CAMHB. The subculture was then transferred to culture tubes in 3 mL aliquots, which were dosed with adjuvant, colistin, or adjuvant plus colistin. One aliquot was not dosed to serve as a control. All samples were then incubated at 37 C with shaking. At 2, 4, 6, 8, and 24-hour time points, 100 μL was taken from each sample and ten-fold diluted in CAMHB up to 7 times. 100 μL of diluted culture was plated on LB (Lennox) agar and incubated at 37 C overnight. The total number of bacterial colonies on each plate was determined using a SphereFlash colony counter (NEUTEC Group Inc.).

Cell line Toxicity: 4T1 cells (ATCC Manassas, Va.) were plated at a density of 1×104 cells/well in 96-well plates in Roswell Park Memorial Institute Media 1640 (RPMI)

(Gibco, Gaithersburg, MD) supplemented with 10% Fetal Bovine Serum (Gibco), 2 mM GlutaMAX (Gibco) and 50 μM 2-mercaptoethanol (Sigma Aldrich, St. Louis, Mo.) and incubated at 37 C under a 5% CO₂ atmosphere in the dark for 18 h. Cell cultures were treated with serial dilutions of compounds in the presence or absence of 1 μg/mL colistin (3 replicates per condition) and incubated for an additional 18 hours. The following control conditions were used: media only (blank), 1% Triton X100 (0% cell viability), 0.5% DMSO (100% cell viability). Each condition was then treated with 10% volume of a 5 mg/mL solution of 3-(4,5-dimethylthiazol-2-yl)-2,5-diphenyltetrazolium bromide (MTT) (Sigma Aldrich) in sterile filtered 1X phosphate buffered saline (PBS) and incubated for 2 h. at 37 C in 5% CO₂, after which the media was aspirated and the resulting formazan crystals were resuspended in 100 μL acidified (4 mM HCl) isopropanol. The 96-well plate was then read at 540 nm on a FLUOstar Optima (BMG Labtech Cary, NC) microplate reader. Cell viability was calculated as a percentage using the two previously mentioned controls.

### Example 3

### Screening Data

TABLE 4
Initial screen of IMD-0354 analogues for colistin
resistance suppression.

| | KP B9 | | AB 4106 | |
|---|---|---|---|---|
| | | Colistin MIC | | Colistin MIC |
| | MIC | (μg/mL) | MIC | (μg/mL) |
| Compound | (μM) | (fold reduction)^{a} | (μM) | (fold reduction) |
| — | | 512 | — | 2048 |
| IMD-0354 | 50 | 0.5 (1024) | 50 | 2 (1024) |
| 1 | >200 | >64 (≤4) | >200 | >64 (≤16) |
| 2 | >200 | >64 (≤4) | >200 | >64 (≤16) |
| 3 | >200 | 0.5 (1024) | >200 | 4 (512) |
| 4 | >200 | >64 (≤4) | >200 | >64 (≤16) |
| 5 | 200 | 1 (512) | 100 | 4 (512) |
| 6 | >200 | 0.5 (1024) | >200 | 4 (512) |
| 7 | >200 | >64 (≤4) | >200 | >64 (≤16) |
| 8 | >200 | 0.5 (1024) | >200 | 2 (1024) |
| 9 | 25 | 0.25 (2048) | >100 | 2 (1024) |
| 10 | >200 | 0.5 (1024) | >200 | 0.5 (4096) |
| 11 | >200 | 16 (32) | >200 | 8 (256) |
| 12 | >200 | 0.5 (1024) | >200 | 4 (512) |
| 13 | >200 | 4 (128) | >200 | 4 (512) |
| 14 | 100 | 1 (512) | 100 | 4 (512) |
| 15 | >200 | 0.25 (2048) | >200 | 1 (2048) |
| 16 | 200 | 2 (256) | >200 | >64 (≤16) |
| 17 | >200 | 0.5 (1024) | >200 | 2 (1024) |
| 18 | 50 | 0.5 (1024) | >200 | 2 (1024) |
| 19 | >200 | >64 (≤4) | >200 | >64 (≤16) |
| 20 | >200 | 1 (512) | >200 | 2 (1024) |
| 21 | 200 | 0.25 (2048) | >200 | 2 (1024) |
| 22 | >200 | 0.25 (2048) | >200 | 2 (1024) |
| 23 | >200 | 0.25 (2048) | >200 | 2 (1024) |
| 24 | >200 | 0.5 (1024) | >200 | 0.5 (4096) |
| 25 | 100 | 0.25 (2048) | 50 | 1 (2048) |
| 26 | >200 | 0.25 (2048) | >200 | 1 (2048) |
| 27 | >200 | 1 (512) | >200 | 8 (256) |
| 28 | >200 | 0.5 (1024) | >200 | 2 (1024) |
| 29 | >200 | 0.5 (1024) | >200 | 1 (2048) |
| 30 | >200 | 4 (128) | >200 | 64 (32) |
| 31 | >200 | 16 (32) | >200 | 64 (32) |
| 32 | >200 | 8 (64) | >200 | 64 (32) |
| 33 | >200 | 2 (256) | >200 | 16 (128) |
| 34 | >200 | >64 (≤4) | >200 | >64 (≤16) |
| 35 | >200 | 1 (512) | >200 | 64 (32) |
| 36 | >200 | >64 (≤4) | >200 | >64 (≤16) |
| 37 | >200 | 0.25 (2048) | >200 | 1 (2048) |
| 38 | >200 | >64 (≤4) | >200 | >64 (≤16) |
| 39 | >200 | 4(128) | >200 | 8 (256) |
| 40 | >200 | 0.5 (1024) | >200 | 2 (1024) |
| 41 | >200 | 2 (256) | >200 | 16 (128) |
| 42 | 25 | 0.5 (1024) | 200 | 8 (256) |
| 43 | >200 | 0.125 (4096) | >200 | 2 (1024) |
| 44 | >200 | >64 (≤4) | >200 | >64 (≤16) |
| 45 | >200 | 0.5 (1024) | >200 | 2 (1024) |
| 46 | >200 | 0.25 (2048) | >200 | 4 (512) |
| 47 | >200 | 0.5 (1024) | >200 | 2 (1024) |
| 48 | >200 | 1 (512) | >200 | 16 (128) |
| 49 | >200 | 0.25 (2048) | 200 | 1 (2048) |
| 50 | >200 | 0.25 | >200 | 4 (512) |
| 51 | >200 | 0.125 | >200 | 1 (2048) |
| 52 | >200 | 0.5 | >200 | 1 (2048) |
| 53 | >200 | 1 | >200 | 8 (256) |
| 54 | 100 | 1 | 200 | 2 (1024) |
| 55 | >200 | 0.5 | >200 | 1 (2048) |
| 56 | >200 | 0.5 | >200 | 1 (2048) |
| 57 | >200 | 1 | >200 | 8 (256) |
| 58 | >200 | >64 (≤4) | >200 | >64 (≤16) |
| 59 | >200 | 32 | >200 | >64 (≤16) |
| 60 | 50 | 0.5 | 50 | 1 (2048) |
| 61 | >200 | >64 (≤4) | >200 | >64 (≤16) |
| 62 | 50 | 1 | 100 | 4 (512) |

^{a}All compounds tested at 5 μM

TABLE 5
Activity of compounds identified in initial screen against colistin resistant isolate panel.

| | KP | KP | KP | AB | AB | AB | AB | AB |
|---|---|---|---|---|---|---|---|---|
| Compound | C3 | A5 | F2210291^{mcr-1} | 3941 | 3942 | 4112 | 4119 | 17978^{mcr-1} |
| | 128 | 2048 | 16 | 1024 | >2048 | 2048 | 1024 | 64 |
| IMD-0354 | 0.5 | 8 | 0.5 | 2 | 2 | 2 | 4 | 0.5 |
| 3 | 4 | >64 | 1 | 8 | 8 | 8 | 4 | 0.5 |
| 5 | 2 | 4 | 0.5 | 8 | 8 | 8 | 4 | N.T.^{b} |
| 6 | 1 | 2 | 0.5 | 4 | 4 | 4 | 4 | 0.5 |
| 8 | 1 | 4 | 1 | 2 | 2 | 2 | 2 | 0.5 |
| 9 | 1 | 1 | 0.5 | 2 | 2 | 2 | 2 | 0.5 |
| 10 | 0.5 | 2 | 0.5 | 1 | 1 | 1 | 1 | 1 |
| 12 | 2 | >64 | 0.5 | 4 | 4 | 4 | 2 | 0.5 |
| 13 | N.T. | N.T. | N.T. | 16 | 32 | 8 | 2 | 4 |
| 14 | 2 | 4 | 1 | 8 | 4 | 4 | 4 | 1 |
| 15 | 0.5 | 4 | 0.25 | 2 | 2 | 1 | 2 | 0.25 |
| 17 | 1 | 4 | 0.25 | 4 | 4 | 4 | 4 | 0.25 |
| 18 | 1 | 2 | 0.5 | 2 | 2 | 2 | 2 | 0.5 |
| 20 | 0.5 | 1 | 0.5 | 4 | 4 | 4 | 4 | 0.25 |
| 21 | 0.25 | 0.5 | 0.5 | 2 | 2 | 2 | 4 | 0.25 |
| 22 | 0.5 | 0.5 | 0.25 | 2 | 2 | 2 | 2 | 0.25 |
| 23 | 0.5 | 0.5 | 0.125 | 2 | 2 | 2 | 1 | 0.5 |
| 24 | 0.5 | 4 | 0.25 | 1 | 0.5 | 0.25 | 0.125 | 0.25 |
| 25 | 0.5 | 4 | 0.5 | 1 | 1 | 1 | 1 | 0.5 |
| 26 | 0.5 | 1 | 0.5 | 2 | 2 | 2 | 2 | 0.25 |
| 27 | 2 | 8 | 0.5 | N.T. | N.T. | N.T. | N.T. | N.T. |
| 28 | 0.5 | 2 | 0.25 | 2 | 2 | 2 | 1 | 0.25 |
| 29 | 1 | 1 | 0.25 | 1 | 1 | 1 | 2 | 0.125 |
| 35 | 2 | 8 | 1 | N.T. | N.T. | N.T. | N.T. | N.T. |
| 37 | 0.25 | 0.5 | 0.25 | 2 | 2 | 2 | 1 | 0.25 |
| 40 | 0.5 | 2 | 0.5 | 8 | 4 | 4 | 4 | 0.25 |
| 42 | 0.5 | 2 | 0.25 | N.T. | N.T. | N.T. | N.T. | N.T. |
| 43 | 0.25 | 1 | 0.25 | 2 | 2 | 2 | 2 | 0.125 |
| 45 | 0.25 | 0.5 | 0.5 | 2 | 2 | 2 | 1 | 0.25 |
| 46 | 0.5 | 1 | 0.25 | 2 | 4 | 4 | 4 | 0.5 |
| 47 | 0.25 | 1 | 0.25 | 2 | 2 | 2 | 1 | 0.5 |
| 48 | 2 | 8 | 0.5 | N.T. | N.T. | N.T. | N.T. | N.T. |
| 49 | 0.5 | 4 | 0.25 | 2 | 2 | 2 | 1 | 0.25 |
| 50 | 0.5 | 1 | 0.5 | 4 | 4 | 2 | 4 | 0.25 |
| 51 | 0.25 | 0.25 | 0.25 | 1 | 1 | 1 | 0.5 | 0.125 |
| 52 | 0.5 | 1 | 0.25 | 1 | 1 | 1 | 2 | 0.25 |
| 53 | 1 | 8 | 0.5 | N.T. | N.T. | N.T. | N.T. | N.T. |
| 54 | 0.5 | 1 | 0.5 | 4 | 2 | 2 | 2 | 0.5 |
| 55 | 0.5 | 1 | 0.5 | 2 | 2 | 1 | 1 | 0.5 |
| 56 | 0.5 | 1 | 0.25 | 2 | 1 | 2 | 2 | 0.125 |
| 57 | 4 | 8 | 0.5 | N.T. | N.T. | N.T. | N.T. | N.T. |
| 60 | 1 | 2 | 0.5 | 2 | 2 | 2 | 2 | 0.25 |
| 62 | 1 | 1 | 0.5 | 8 | 8 | 4 | 4 | 0.5 |

^{a}All compounds tested at 5 μM.
^{b}N.T. not tested

TABLE 6
Dose response data for active analogues against KP B9.

| | | Colistin MIC |
|---|---|---|
| | Concentration | (μg/mL) |
| Compound | (μM) | (fold reduction) |
| IMD-0354 | 5 | 0.5 (1024) |
| | 3 | 0.5 (1024) |
| | 1 | 4 (128) |
| 3 | 5 | 0.5 (1024) |
| | 3 | 1 (512) |
| | 1 | >64 (≤4) |
| 5 | 5 | 1 (512) |
| | 3 | 2 (256) |
| | 1 | 16 (32) |
| 6 | 5 | 0.5 (1024) |
| | 3 | 0.5 (1024) |
| | 1 | 4 (128) |
| 8 | 5 | 0.5 (1024) |
| | 3 | 1 (512) |
| | 1 | 8 (64) |
| 9 | 5 | 0.25 (2048) |
| | 3 | 0.5 (1024) |
| | 1 | 2 (256) |
| 10 | 5 | 0.5 (1024) |
| | 3 | 1 (512) |
| | 1 | 2 (256) |
| 12 | 5 | 0.5 (1024) |
| | 3 | 1 (512) |
| | 1 | >64 (≤4) |
| 14 | 5 | 1 (512) |
| | 3 | 2 (256) |
| | 1 | 16 (32) |
| 15 | 5 | 0.25 (2048) |
| | 3 | 0.5 (1024) |
| | 1 | 1 (512) |
| | 0.5 | 4 (128) |
| 17 | 5 | 0.5 (1024) |
| | 3 | 0.5 (1024) |
| | 1 | 2 (256) |
| 18 | 5 | 0.5 (1024) |
| | 3 | 2 (256) |
| | 1 | 4 (128) |
| 20 | 5 | 0.5 (1024) |
| | 3 | 0.5 (1024) |
| | 1 | 16 (32) |
| 21 | 5 | 0.25 (2048) |
| | 3 | 0.5 (1024) |
| | 1 | 1 (512) |
| 22 | 5 | 0.25 (2048) |
| | 3 | 0.5 (1024) |
| | 1 | 0.5 (1024) |
| | 0.5 | 4 (128) |
| 23 | 5 | 0.25 (2048) |
| | 3 | 0.5 (1024) |
| | 1 | 0.5 (1024) |
| | 0.5 | 1 (512) |
| | 0.25 | 4 (128) |
| 24 | 5 | 0.5 (1024) |
| | 3 | 1 (512) |
| | 1 | 2 (256) |
| 25 | 5 | 0.25 (2048) |
| | 3 | 1 (512) |
| | 1 | 1 (512) |
| | 0.5 | 16 (32) |
| 26 | 5 | 0.25 (2048) |
| | 3 | 0.5 (1024) |
| | 1 | 0.5 (1024) |
| | 0.5 | 8 (64) |
| 28 | 5 | 0.5 (1024) |
| | 3 | 0.5 (1024) |
| | 1 | 0.5 (1024) |
| | 0.5 | 4 (128) |
| 29 | 5 | 0.5 (1024) |
| | 3 | 0.5 (1024) |
| | 1 | 0.5 (1024) |
| | 0.5 | 0.5 (1024) |
| | 0.25 | 1 (512) |
| | 0.125 | >64 (≤4) |
| 35 | 5 | 1 (512) |
| | 3 | 2 (256) |
| | 1 | >64 (≤4) |
| 37 | 5 | 0.25 (2048) |
| | 3 | 0.5 (1024) |
| | 1 | 0.5 (1024) |
| | 0.5 | 64 (32) |
| 40 | 5 | 0.5 (1024) |
| | 3 | 1 (512) |
| | 1 | 16 (32) |
| 42 | 5 | 0.5 (1024) |
| | 3 | 0.5 (1024) |
| | 1 | >64 |
| 43 | 5 | 0.125 (4096) |
| | 3 | 1 (512) |
| | 1 | 2 (256) |
| 45 | 5 | 0.5 (1024) |
| | 3 | 0.5 (1024) |
| | 1 | 4 (128) |
| 46 | 5 | 0.25 (2048) |
| | 3 | 2 (256) |
| | 1 | 8 (64) |
| 47 | 5 | 0.5 (1024) |
| | 3 | 1 (512) |
| | 1 | 2 (256) |
| 48 | 5 | 1 (512) |
| | 3 | 2 (256) |
| | 1 | >64 (≤4) |
| 49 | 5 | 0.25 (2048) |
| | 3 | 0.5 (1024) |
| | 1 | 1 (512) |
| | 0.5 | 4 (128) |
| 50 | 5 | 0.25 (2048) |
| | 3 | 0.5 (1024) |
| | 1 | 2 (256) |
| 51 | 5 | 0.125 (4096) |
| | 3 | 0.25 (2048) |
| | 1 | 0.5 (1024) |
| | 0.5 | 1 (512) |
| | 0.25 | 16 (32) |
| 52 | 5 | 0.5 (1024) |
| | 3 | 0.5 (1024) |
| | 1 | 1 (512) |
| | 0.5 | 1 (512) |
| | 0.25 | 16 (32) |
| 53 | 5 | 1 (512) |
| | 3 | 1 (512) |
| | 1 | >64 (≤4) |
| 54 | 5 | 1 (512) |
| | 3 | 2 (256) |
| | 1 | 4 (128) |
| 55 | 5 | 0.5 (1024) |
| | 3 | 2 (256) |
| | 1 | 2 (256) |
| 56 | 5 | 0.5 (1024) |
| | 3 | 0.5 (1024) |
| | 1 | 4 (128) |
| 57 | 5 | 1 (512) |
| | 3 | 1 (512) |
| | 1 | >64 (≤4) |
| 60 | 5 | 0.5 (1024) |
| | 3 | 0.5 (1024) |
| | 1 | 2 (256) |
| 62 | 5 | 1 (512) |
| | 3 | 1 (512) |
| | 1 | 8 (64) |

TABLE 7
Dose response data for active analogues against AB 4106.

| | | Colistin MIC |
|---|---|---|
| | Concentration | (μg/mL) |
| Compound | (μM) | (fold reduction) |
| — | — | 2048 μg/mL |
| IMD-0354 | 5 | 2 (1024) |
| | 3 | 4 (512) |
| | 1 | 8 (256) |
| 3 | 5 | 4 (512) |
| | 3 | 8 (256) |
| | 1 | >64 (≤16) |
| 5 | 5 | 4 (512) |
| | 3 | 8 (256) |
| | 1 | >64 (≤16) |
| 6 | 5 | 4 (512) |
| | 3 | 4 (512) |
| 8 | 5 | 2 (1024) |
| | 3 | 2 (1024) |
| | 1 | 32 (64) |
| 9 | 5 | 2 (1024) |
| | 3 | 2 (1024) |
| | 1 | 16 (128) |
| 10 | 5 | 0.5 (4096) |
| | 3 | 2 (1024) |
| | 1 | 16 (128) |
| 12 | 5 | 4 (512) |
| | 3 | 8 (256) |
| | 1 | >64 (≤16) |
| 13 | 5 | 4 (512) |
| | 3 | 8 (256) |
| | 1 | 64 (32) |
| 14 | 5 | 4 (512) |
| | 3 | 4 (512) |
| | 1 | 64 (32) |
| 15 | 5 | 1 (2048) |
| | 3 | 4 (512) |
| | 1 | 16 (128) |
| 17 | 5 | 2 (1024) |
| | 3 | 4 (512) |
| | 1 | 16 (128) |
| 18 | 5 | 2 (1024) |
| | 3 | 2 (1024) |
| | 1 | 8 (256) |
| 20 | 5 | 2 (1024) |
| | 3 | 4 (512) |
| | 1 | 128 (16) |
| 21 | 5 | 2 (1024) |
| | 3 | 4 (512) |
| | 1 | 32 (64) |
| 22 | 5 | 2 (1024) |
| | 3 | 2 (1024) |
| | 1 | 4 (512) |
| 23 | 5 | 2 (1024) |
| | 3 | 2 (1024) |
| | 1 | 8 (256) |
| 24 | 5 | 0.5 (4096) |
| | 3 | 1 (2048) |
| | 1 | 4 (512) |
| 25 | 5 | 1 (2048) |
| | 3 | 2 (1024) |
| | 1 | 4 (512) |
| 26 | 5 | 1 (2048) |
| | 3 | 2 (1024) |
| | 1 | 16 (128) |
| 28 | 5 | 2 (1024) |
| | 3 | 2 (1024) |
| | 1 | 8 (256) |
| 29 | 5 | 1 (2048) |
| | 3 | 2 (1024) |
| | 1 | 4 (512) |
| 37 | 5 | 1 (2048) |
| | 3 | 2 (1024) |
| | 1 | 16 (128) |
| 40 | 5 | 2 (1024) |
| | 3 | 8 (256) |
| | 1 | >64 (≤16) |
| 43 | 5 | 2 (1024) |
| | 3 | 4 (512) |
| | 1 | 16 (128) |
| | 5 | 2 (1024) |
| 45 | 3 | 4 (512) |
| | 1 | 8 (256) |
| 46 | 5 | 4 (512) |
| | 3 | 8 (256) |
| | 1 | 64 (32) |
| 47 | 5 | 2 (1024) |
| | 3 | 2 (1024) |
| | 1 | 8 (256) |
| 49 | 5 | 1 (2048) |
| | 3 | 2 (1024) |
| | 1 | 8 (256) |
| 50 | 5 | 4 (512) |
| | 3 | 8 (256) |
| | 1 | 32 (64) |
| 51 | 5 | 1 (2048) |
| | 3 | 1 (2048) |
| | 1 | 4 (512) |
| 52 | 5 | 1 (2048) |
| | 3 | 2 (1024) |
| | 1 | 8 (256) |
| 54 | 5 | 2 (1024) |
| | 3 | 4 (512) |
| | 1 | 32 (64) |
| 55 | 5 | 1 (2048) |
| | 3 | 2 (1024) |
| | 1 | 8 (256) |
| 56 | 5 | 1 (2048) |
| | 3 | 2 (1024) |
| | 1 | 8 (256) |
| 60 | 5 | 1 (2048) |
| | 3 | 4 (512) |
| | 1 | 16 (128) |
| 62 | 5 | 4 (512) |
| | 3 | 8 (256) |
| | 1 | 64 (32) |

### Example 4

### Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel l | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl mono stearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 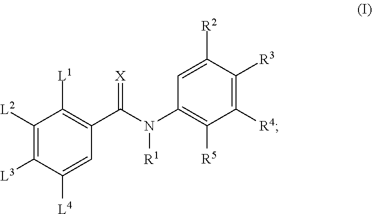
wherein
L¹ is OH, H, SH, NH₂, or —O(C═O)(C₁-C₈)alkyl wherein the moiety (C₁-C₈)alkyl is interrupted optionally with one or more heteroatoms;
L² is H, OH, or halo;
L³ is H, OH, or CF₃;
L⁴ is halo, H, or OH;
R¹ is H or —(C₁-C₆)alkyl;
R² is H, F, Br, I, CF₃, or —(C₁-C₆)alkyl;
R³ is CF₃, H, or halo;
R⁴ is F, Br, I, H, CF₃, NH₂, NO₂, or —(C₁-C₆)alkyl;
R⁵ is H, or CF₃; and
X is O or S;
wherein at least one of L¹, L², L³ and L⁴ is OH or —O(C═O)(C₁-C₈)alkyl; and
wherein at least two of R², R³, R⁴ and R⁵ are not H.

2. The compound of claim 1 wherein R¹ is H and X is O.

3. The compound of claim 1 wherein the compound of Formula I is represented by Formula Ia or Ib:

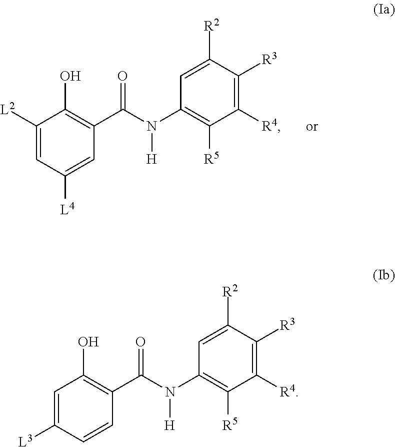

4. The compound of claim 1 wherein the compound of Formula I is represented by Formula IIa or IIb:

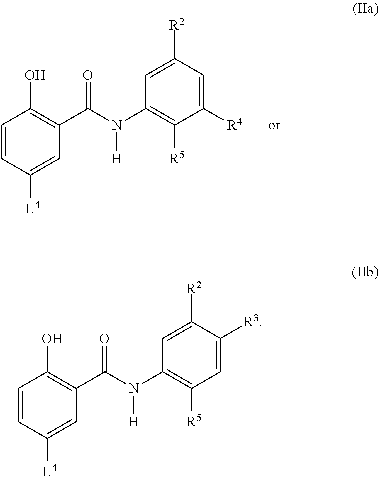

5. The compound of claim 1 wherein the compound of Formula I is represented by Formula IIa or IIIb:

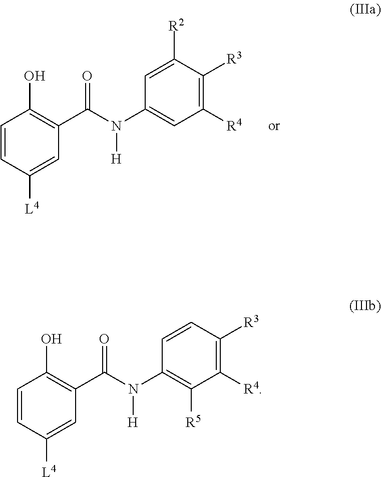

6. The compound of claim 1 wherein the compound of Formula I is represented by Formula IV:

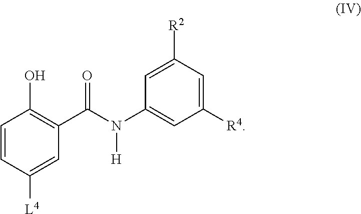

7. The compound of claim 1 wherein the compound of Formula I is represented by Formula Va or Vb:

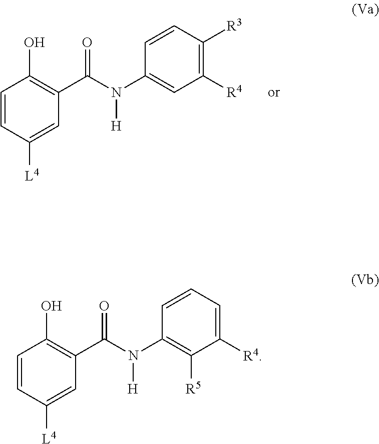

8. The compound of claim 1 wherein the compound of Formula I is represented by Formula VIa or VIb:

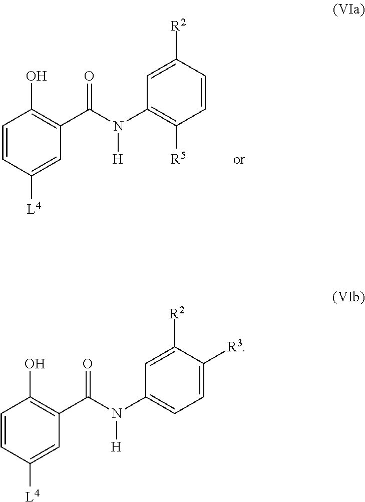

9. The compound of claim 1 wherein at least one other of L¹, L², L³ and L⁴ is not H.

10. The compound of claim 1 wherein L⁴ is halo.

11. The compound of claim 1 wherein at least two of R², R³, R⁴ and R⁵ are not H.

12. The compound of claim 1 wherein at least two of R², R³, R⁴ and R⁵ comprises a halo substituent; or at least two of R², R³, R⁴ and R⁵ are halo.

13. (canceled)

14. The compound of claim 1 wherein at least two of R², R³ and R⁴ are fluoro; or
at least two of R², R³ and R⁴ are bromo; or
at least two of R², R³, R⁴ and R⁵ are CF₃; or
at least one of R², R³, R⁴ and R⁵ is halo and the other is CF₃.

15. (canceled)

16. (canceled)

17. The compound of claim 1 comprising at least one iodo substituent.

18. (canceled)

19. (canceled)

20. The compound of claim 1 wherein the compound is:

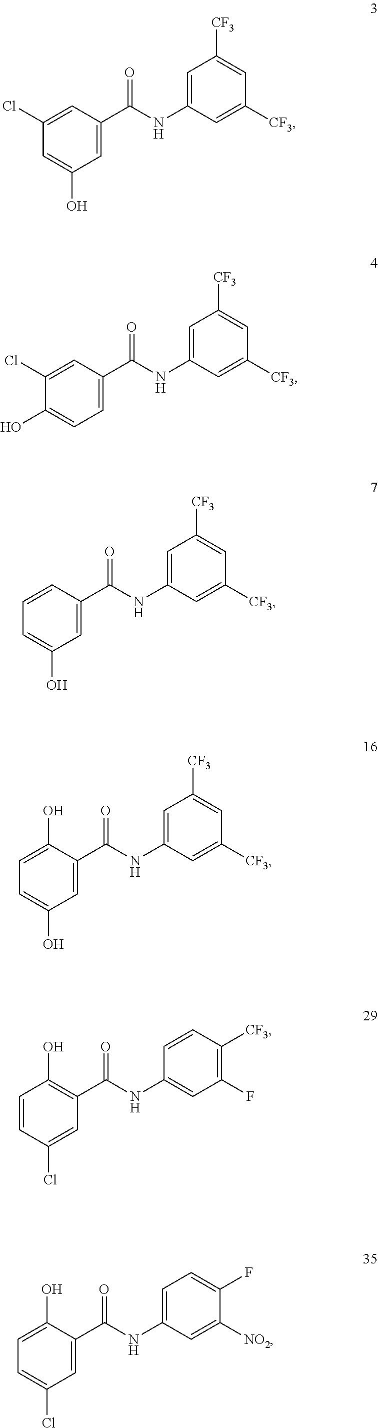 [SMILES: O=C(Nc1cc(C(F)(F)F)cc(C(F)(F)F)c1)c1cc(O)cc(Cl)c1 ; O=C(Nc1cc(C(F)(F)F)cc(C(F)(F)F)c1)c1ccc(O)c(Cl)c1 ; O=C(Nc1cc(C(F)(F)F)cc(C(F)(F)F)c1)c1cccc(O)c1 ; O=C(Nc1cc(C(F)(F)F)cc(C(F)(F)F)c1)c1cc(O)ccc1O ; O=C(Nc1ccc(C(F)(F)F)c(F)c1)c1cc(Cl)ccc1O ; O=C(Nc1ccc(F)c([N+](=O)[O-])c1)c1cc(Cl)ccc1O]

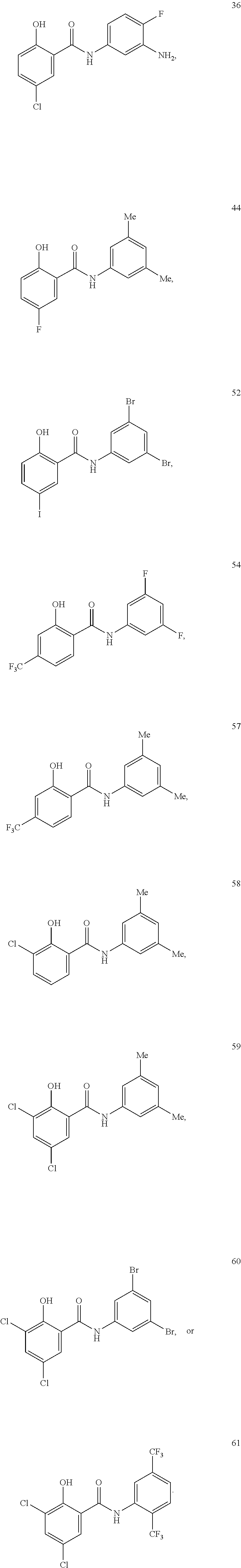

21. The compound of claim 1 wherein the compound is:

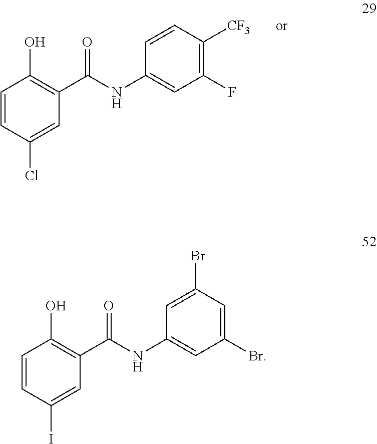 [SMILES: O=C(Nc1ccc(C(F)(F)F)c(F)c1)c1cc(Cl)ccc1O ; O=C(Nc1cc(Br)cc(Br)c1)c1cc(I)ccc1O]

22. A composition comprising the compound of claim 1 and a pharmaceutically acceptable buffer, carrier, diluent, or excipient.

23. A method for treating a bacterial infection in a subject in need thereof comprising administering to the subject, concurrently or sequentially, a therapeutically effective dose of an adjuvant and a therapeutically effective dose of an antibiotic, wherein the adjuvant is a compound of Formula I: 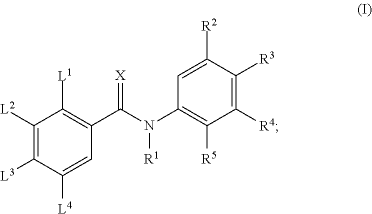
wherein
L¹ is OH, H, SH, NH₂, or —O(C═O)(C₁-C₈)alkyl wherein the moiety (C₁-C₈)alkyl is interrupted optionally with one or more heteroatoms;
L² is H, OH, or halo;
L³ is H, OH, or CF₃;
L⁴ is halo, H, or OH;
R¹ is H or —(C₁-C₆)alkyl;
R² is H, halo, CF₃, or —(C₁-C₆)alkyl;
R³ is CF₃, H, or halo;
R⁴ is halo, H, CF₃, NH₂, NO₂, or —(C₁-C₆)alkyl;
R⁵ is H, halo, or CF₃; and
X is O or S;
wherein at least one of L¹, L², L³ and L⁴ is OH or —O(C═O)(C₁-C₈)alkyl; and
wherein at least one of R², R³, R⁴ and R⁵ is not H; and
the bacterial infection is thereby treated.

24. The method of claim 23 wherein the adjuvant is: 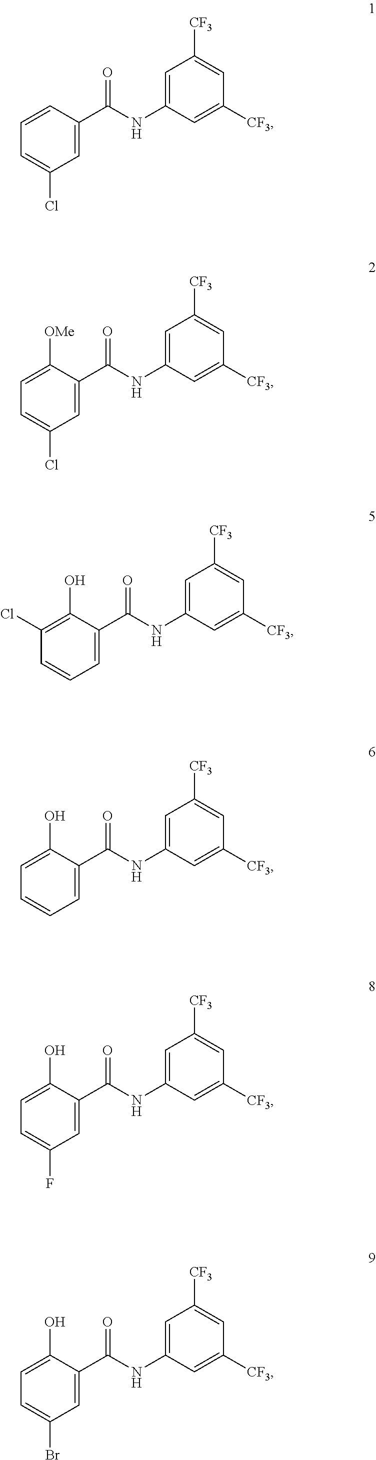 [SMILES: O=C(Nc1cc(C(F)(F)F)cc(C(F)(F)F)c1)c1cccc(Cl)c1 ; COc1ccc(Cl)cc1C(=O)Nc1cc(C(F)(F)F)cc(C(F)(F)F)c1 ; O=C(Nc1cc(C(F)(F)F)cc(C(F)(F)F)c1)c1cccc(Cl)c1O ; O=C(Nc1cc(C(F)(F)F)cc(C(F)(F)F)c1)c1ccccc1O ; O=C(Nc1cc(C(F)(F)F)cc(C(F)(F)F)c1)c1cc(F)ccc1O ; O=C(Nc1cc(C(F)(F)F)cc(C(F)(F)F)c1)c1cc(Br)ccc1O] 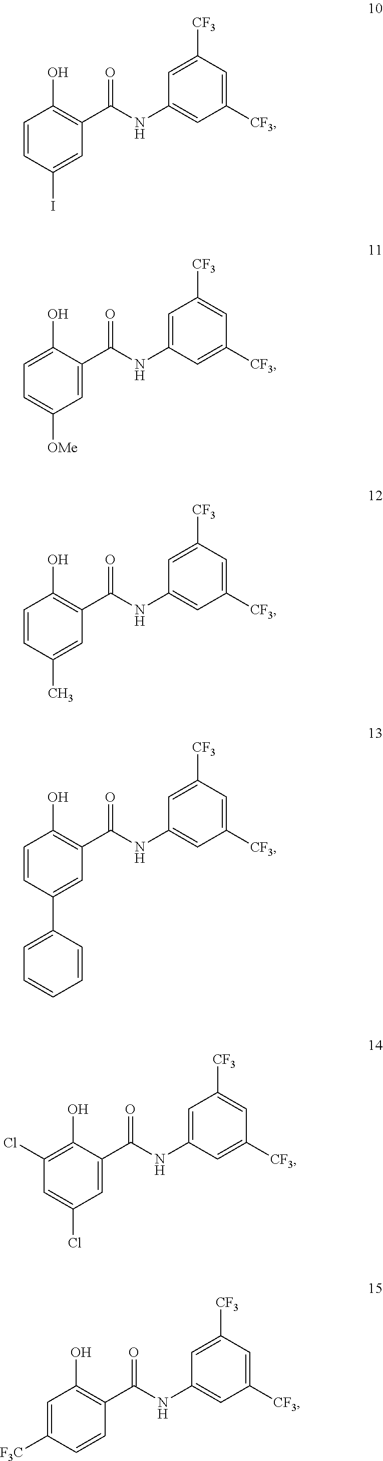 [SMILES: O=C(Nc1cc(C(F)(F)F)cc(C(F)(F)F)c1)c1cc(I)ccc1O ; COc1ccc(O)c(C(=O)Nc2cc(C(F)(F)F)cc(C(F)(F)F)c2)c1 ; Cc1ccc(O)c(C(=O)Nc2cc(C(F)(F)F)cc(C(F)(F)F)c2)c1 ; O=C(Nc1cc(C(F)(F)F)cc(C(F)(F)F)c1)c1cc(-c2ccccc2)ccc1O ; O=C(Nc1cc(C(F)(F)F)cc(C(F)(F)F)c1)c1cc(Cl)cc(Cl)c1O ; O=C(Nc1cc(C(F)(F)F)cc(C(F)(F)F)c1)c1ccc(C(F)(F)F)cc1O] 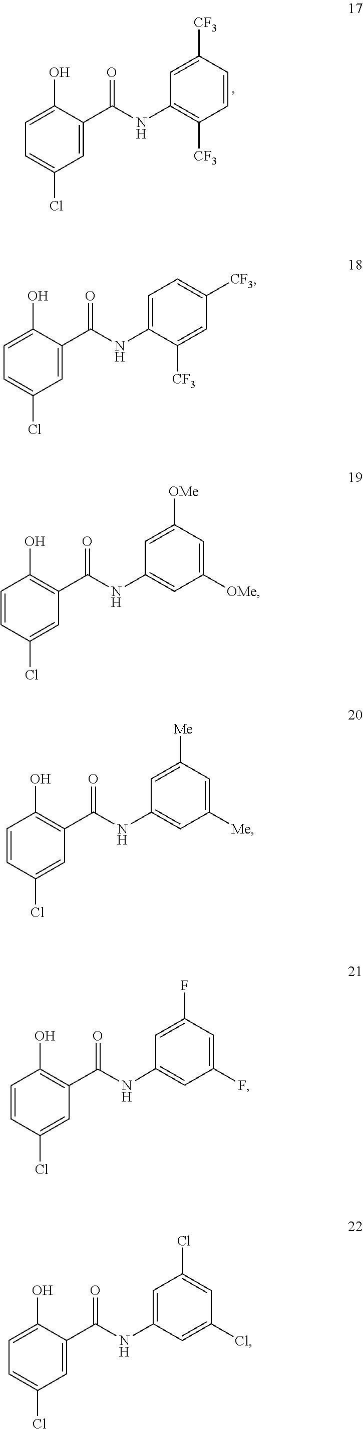 [SMILES: O=C(Nc1cc(C(F)(F)F)ccc1C(F)(F)F)c1cc(Cl)ccc1O ; O=C(Nc1ccc(C(F)(F)F)cc1C(F)(F)F)c1cc(Cl)ccc1O ; COc1cc(NC(=O)c2cc(Cl)ccc2O)cc(OC)c1 ; Cc1cc(C)cc(NC(=O)c2cc(Cl)ccc2O)c1 ; O=C(Nc1cc(F)cc(F)c1)c1cc(Cl)ccc1O ; O=C(Nc1cc(Cl)cc(Cl)c1)c1cc(Cl)ccc1O] 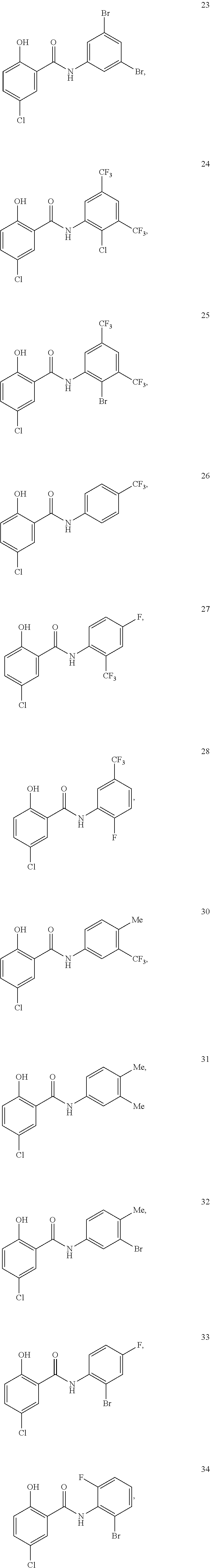 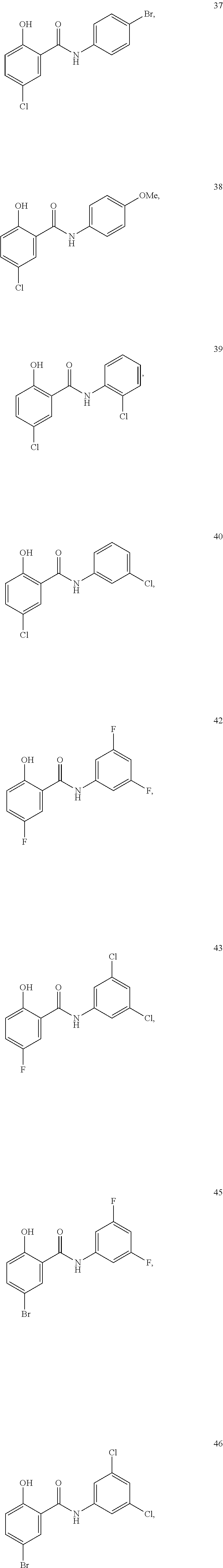 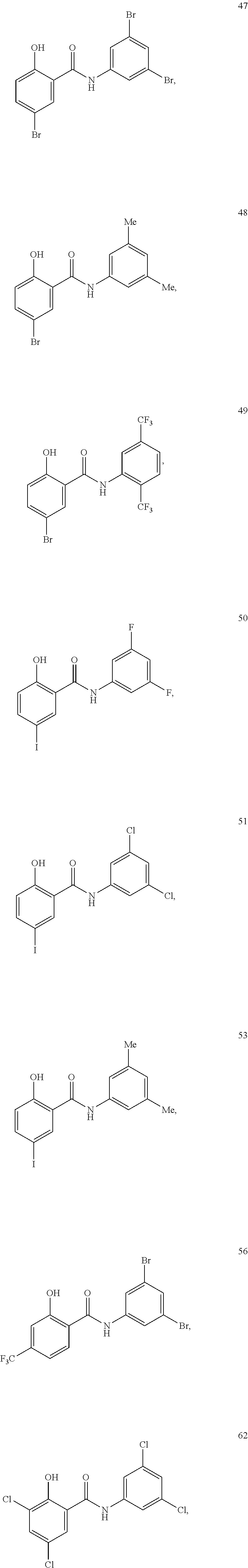
or a pharmaceutical composition thereof.

25. (canceled)

26. The method of claim 23 wherein the antibiotic is Colistin.

27. The method of claim 23 wherein the bacterial infection is a multidrug-resistant Gram-negative bacterial infection.

28. The method of claim 23 wherein the subject has a systemic concentration of the adjuvant of about 0.01 micromolar to about 10 micromolar and/or the subject has a systemic concentration of Colistin of about 0.1 microgram/milliliter to about 50 microgram/milliliter.

29. (canceled)
